(19) United States
(12) Patent Application Publication
(10) Pub. No.: US 2017/0281642 A1
(43) Pub. Date: Oct. 5, 2017

(54) METHODS OF TREATING ACUTE MYELOID LEUKEMIA OR ACUTE LYMPHOID LEUKEMIA USING PHARMACEUTICAL COMPOSITIONS CONTAINING THIENOTRIAZOLODIAZEPINE COMPOUNDS

(71) Applicant: ONCOETHIX GmbH, Lucerne (CH)

(72) Inventor: Hervé Dombret, Paris (FR)

(73) Assignee: ONCOETHIX GmbH, Lucerne (CH)

(21) Appl. No.: 15/507,496

(22) PCT Filed: Aug. 28, 2015
(86) PCT No.: PCT/EP2015/069754

§ 371 Date: Feb. 28, 2017

Related U.S. Application Data
(60) Provisional application No. 62/043,063, filed on Aug. 28, 2014.

(51) Int. Cl. A61K 31/551; A61K 47/38; A61K 9/10

(52) U.S. Cl. CPC A61K 31/551; A61K 9/10; A61K 47/38

(57) ABSTRACT

A method of treating acute myeloid leukemia or acute lymphoid leukemia in a mammal comprising the step of: administering a pharmaceutically acceptable amount of a thienotriazolodiazepine compound represented by the following Formula (1): wherein R¹ is alkyl having a carbon number of 1-4, R² is a hydrogen atom; a halogen atom; or alkyl having a carbon number of 1-4 optionally substituted by a halogen atom or a hydroxyl group, R³ is a halogen atom; phenyl optionally substituted by a halogen atom, alkyl having a carbon number of 1-4, alkoxy having a carbon number of 1-4 or cyano; —NR⁵—(CH₂)ₘ—R⁶ wherein R⁵ is a hydrogen atom or alkyl having a carbon number of 1-4, m is an integer of 0-4, and R6 is phenyl or pyridyl optionally substituted by a halogen atom; or —NR⁷—CO—(CH₂)ₙ—R⁸ wherein R⁷ is a hydrogen atom or alkyl having a carbon number of 1-4, n is an integer of 0-2, and R⁸ is phenyl or pyridyl optionally substituted by a halogen atom, and R⁴ is —(CH₂)ₐ—CO—NH—R⁹ wherein a is an integer of 1-4, and R⁹ is alkyl having a carbon number of 1-4; hydroxyalkyl having a carbon number of 1-4; alkoxy having a carbon substituted by alkyl having a carbon number of 1-4, alkoxy having a carbon number of 1-4, amino or a hydroxyl group or —(CH₂)_{b}—COOR¹⁰ wherein b is an integer of 1-4, and R¹⁰ is alkyl having a carbon number of 1-4, or a pharmaceutically acceptable salt thereof or a hydrate or solvate thereof

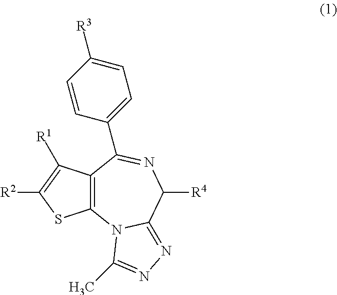

### FIELD OF INVENTION

The present disclosure is concerned with methods of treatment, particularly methods of treating acute myeloid leukemia or acute lymphoid leukemia in a mammal using thienotriazolodiazepine compounds.

### BACKGROUND OF THE INVENTION

Acute myeloid and acute lymphoid leukemias (AML and ALL) constitute a genetically complex and heterogeneous group of tumors associated with maturation arrest, expansion of abnormal hematopoietic progenitors, and abnormal remodeling of chromatin.

### BRIEF SUMMARY OF THE INVENTION

In an aspect, the present invention provides a method of treating acute myeloid leukemia or acute lymphoid leukemia in a mammal using the thienotriazolodiazepine compounds described herein. In some embodiments, the method of treating acute myeloid leukemia or acute lymphoid leukemia in a mammal comprises administering a pharmaceutically acceptable amount of a thienotriazolodiazepine compound represented by the thienotriazolodiazepine compound of Formula (1)

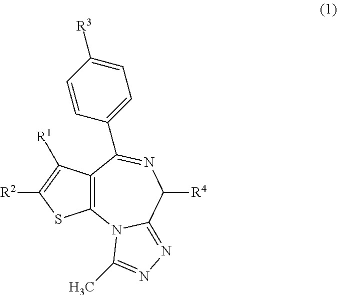

wherein R¹ is alkyl having a carbon number of 1-4,
R² is a hydrogen atom; a halogen atom; or alkyl having a carbon number of 1-4 optionally substituted by a halogen atom or a hydroxyl group,
R³ is a halogen atom; phenyl optionally substituted by a halogen atom, alkyl having a carbon number of 1-4, alkoxy having a carbon number of 1-4 or cyano; —NR⁵—(CH₂)ₘ—R⁶ wherein R⁵ is a hydrogen atom or alkyl having a carbon number of 1-4, m is an integer of 0-4, and R⁶ is phenyl or pyridyl optionally substituted by a halogen atom; or —NR⁷—CO—(CH₂)n-R⁸ wherein R⁷ is a hydrogen atom or alkyl having a carbon number of 1-4, n is an integer of 0-2, and R⁸ is phenyl or pyridyl optionally substituted by a halogen atom, and
R⁴ is —(CH₂)ₐ—CO—NH—R⁹ wherein a is an integer of 1-4, and R⁹ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR¹⁰ wherein b is an integer of 1-4, and R¹⁰ is alkyl having a carbon number of 1-4,
or a pharmaceutically acceptable salt thereof or a hydrate or solvate thereof. In some such embodiments the expression of HEXIM1 is upregulated after administration of the thienotriazolodiazepine compound represented by Formula (1).

In some embodiments, Formula (1) is selected from Formula (1A):

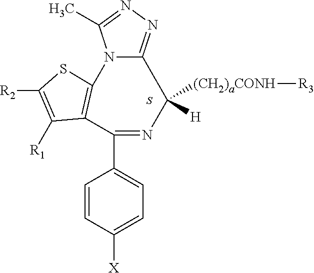

wherein X is a halogen, R¹ is C₁-C₄ alkyl, R² is C₁-C₄ alkyl, a is an integer of 1-4, R³ is C₁-C₄ alkyl, C₁-C₄ hydroxyalkyl, C₁-C₄ alkoxy, phenyl optionally having substituent(s) as defined for R⁹ in Formula (1), or heteroaryl optionally having substituent(s) as defined for R⁹ in Formula (1), a pharmaceutically acceptable salt thereof or a hydrate thereof.

In one embodiment of the method of treating acute myeloid leukemia or acute lymphoid leukemia, the thienotriazolodiazepine compound of Formula (1) is formed as a solid dispersion. In a further embodiment of the present invention, the thienotriazolodiazepine compound is formulated as a solid dispersion comprising an amorphous thienotriazolodiazepine compound and a pharmaceutically acceptable polymer.

In some embodiments, the present disclosure provides for a compound of Formula (1), in particular a compound of Formula (1A), for use in treating acute myeloid leukemia. In some embodiments, the present disclosure provides for a compound of Formula (1), in particular a compound of Formula (1A), for use in treating acute lymphoid leukemia. In an embodiment of the present invention, the thienotriazolodiazepine compound of Formula (1) is formed as a solid dispersion. In a further embodiment of the present invention, the thienotriazolodiazepine compound is formulated as a solid dispersion comprising an amorphous thienotriazolodiazepine compound and a pharmaceutically acceptable polymer.

In one such embodiment, the thienotriazolodiazepine compound is formulated as a solid dispersion comprising an amorphous thienotriazolodiazepine compound and a pharmaceutically acceptable polymer. In one such embodiment the solid dispersion comprises an amorphous thienotriazolodiazepine compound of (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide dihydrate, a pharmaceutically acceptable salt thereof, or a hydrate thereof; and a pharmaceutically acceptable polymer. In another such embodiment, the solid dispersion exhibits an X-ray powder diffraction pattern substantially free of diffraction lines associated with crystalline thienotriazolodiazepine compound of Formula (1). In another such embodiment, the solid dispersion comprises an amorphous thienotriazolodiazepine compound of the Formula (1), a pharmaceutically acceptable salt thereof or a hydrate thereof; and a pharmaceutically acceptable polymer. In one such embodiment, the pharmaceutically acceptable polymer is hydroxypropylmethylcellulose acetate succinate having a thienotriazolodiazepine compound to hydroxypropylmethylcellulose acetate succinate (HPMCAS) weight ratio of 1:3 to 1:1. In still yet another embodiment, the solid dispersion exhibits a single glass transition temperature (Tg) inflection point ranging from about 130° C. to about 140° C.

In one embodiment of the methods of treating acute myeloid leukemia or acute lymphoid leukemia, the thienotriazolodiazepine compound represented by Formula (1) is selected from the group consisting of: (a) (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide or a dihydrate thereof, (b) methyl (S)-{4-(3′-cyanobiphenyl-4-yl)-2,3,9-trimethyl-6H-thieno[3,2-f][1,2,4]tri-azolo[4,3-a][1,4]diazepin-6-yl} acetate, (c) methyl (S)-{2,3,9-trimethyl-4-(4-phenylaminophenyl)-6H-thieno[3,2-f][1,2,4]triazolo[4,3-a][1,4]diazepin-6-yl} acetate; and (d) methyl (S)-{2,3,9-trimethyl-4-[4-(3-phenylpropionylamino)phenyl]-6H-thieno[3,2-f][1,2,4]triazolo[4,3-a][1,4]diazepin-6-yl} acetate.

In one embodiment of the methods of treating acute myeloid leukemia or acute lymphoid leukemia, the thienotriazolodiazepine compound represented by Formula (1) is (S)-2-[4-(4-chlorophenyl)-2,3,9-trimethyl-6H-thieno[3,2-f][1,2,4]triazolo[4,3-a][1,4]diazepin-6-yl]-N-(4-hydroxyphenyl)acetamide dihydrate.

In one embodiment, the thienotriazolodiazepine compound represented by Formula (1) is (S)-2-[4-(4-chlorophenyl)-2,3,9-trimethyl-6H-thieno[3,2-f][1,2,4]triazolo-[4,3-a][1,4]diazepin-6-yl]-N-(4-hydroxyphenyl)acetamide.

It should be understood that any embodiment of the compounds according to Formula (1) described herein may be used in any embodiment of a pharmaceutical composition described herein, unless indicated otherwise. Moreover, any compound or pharmaceutical composition described herein as embodiment of the invention may be used as a medicament, in particular for treating acute myeloid leukemia or acute lymphoid leukemia as described in embodiments herein, unless indicated otherwise.

### BRIEF DESCRIPTION OF THE DRAWINGS

The foregoing summary, as well as the following detailed description of embodiments of the pharmaceutical compositions including thienotriazolodiazepine formulations and the methods of the present invention, will be better understood when read in conjunction with the appended drawings of exemplary embodiments. It should be understood, however, that the invention is not limited to the precise arrangements and instrumentalities shown.

In the drawings:

FIG. 1A illustrates dissolution profile of a comparator formulation comprising a solid dispersion comprising 25% compound (1-1) and Eudragit L100-55.

FIG. 1B illustrates dissolution profile of a comparator formulation comprising a solid dispersion comprising 50% compound (1-1) and Eudragit L100-55.

FIG. 1C illustrates dissolution profile of an exemplary formulation comprising a solid dispersion comprising 25% compound (1-1) and polyvinylpyrrolidone (PVP).

FIG. 1D illustrates dissolution profile of an exemplary formulation comprising a solid dispersion comprising 50% compound (1-1) and PVP.

FIG. 1E illustrates dissolution profile of an exemplary formulation comprising a solid dispersion comprising 25% compound (1-1) and PVP-vinyl acetate (PVP-VA).

FIG. 1F illustrates dissolution profile of an exemplary formulation comprising a solid dispersion comprising 50% compound (1-1) and PVP-VA.

FIG. 1G illustrates dissolution profile of an exemplary formulation comprising a solid dispersion comprising 25% compound (1-1) and hypromellose acetate succinate (HPMCAS-M).

FIG. 1H illustrates dissolution profile of an exemplary formulation comprising a solid dispersion comprising 50% compound (1-1) and HPMCAS-M.

FIG. 1I illustrates dissolution profile of an exemplary formulation comprising a solid dispersion comprising 25% compound (1-1) and hypromellose phthalate (HPMCP-HP55).

FIG. 1J illustrates dissolution profile of an exemplary formulation comprising a solid dispersion comprising 50% compound (1-1) and HMCP-HP55.

FIG. 2A illustrates results of in vivo screening of an exemplary formulation comprising a solid dispersion of 25% compound (1-1) and PVP.

FIG. 2B illustrates results of an in vivo screening of an exemplary formulation comprising a solid dispersion of 25% compound (1-1) and HPMCAS-M.

FIG. 2C illustrates results of an in vivo screening of an exemplary formulation comprising a solid dispersion of 50% compound (1-1) and HPMCAS-M.

FIG. 3 illustrates powder X-ray diffraction profiles of solid dispersions of compound (1-1).

FIG. 4A illustrates modified differential scanning calorimetry trace for a solid dispersion of 25% compound (1-1) and PVP equilibrated under ambient conditions.

FIG. 4B illustrates modified differential scanning calorimetry trace for a solid dispersion of 25% compound (1-1) and HPMCAS-M equilibrated under ambient conditions.

FIG. 4C illustrates modified differential scanning calorimetry trace for a solid dispersion of 50% compound (1-1) and HPMCAS-M equilibrated under ambient conditions.

FIG. 5 illustrates plot of glass transition temperature (Tg) versus relative humidity (RH) for solid dispersions of 25% compound (1-1) and PVP or HMPCAS-M and 50% compound (1-1) and HPMCAS-MG.

FIG. 6 illustrates modified differential scanning calorimetry trace for a solid dispersion of 25% compound (1-1) and PVP equilibrated under 75% relative humidity.

FIGS. 7A and 7B illustrate plasma concentration versus time curves for Compound (1-1) after 1 mg/kg intravenous dosing (solid rectangles) and 3 mg/kg oral dosing as 25% Compound (1-1):PVP (open circles), 25% Compound (1-1):HPMCAS-MG (open triangles), and 50% Compound (1-1):HPMCAS-MG (open inverted triangles). The inset depicts the same data plotted on a semilogarithmic scale.

FIGS. 8A and 8B illustrate plasma concentration versus time curves for Compound (1-1) after 3 mg/kg oral dosing as 25% Compound (1-1): PVP (open circles), 25% Compound (1-1):HPMCAS-MG (open triangles), and 50% Compound (1-1):HPMCAS-MG (open inverted triangles). The inset depicts the same data plotted on a semi-logarithmic scale.

FIG. 9 illustrates a powder X-ray diffraction profile of solid dispersions of compound (1-1) in HPMCAS-MG at time zero of a stability test.

FIG. 10 illustrates a powder X-ray diffraction profile of solid dispersions of compound (1-1) in HPMCAS-MG after 1 month at 40° C. and 75% relative humidity.

FIG. 11 illustrates a powder X-ray diffraction profile of solid dispersions of compound (1-1) in HPMCAS-MG after 2 months at 40° C. and 75% relative humidity.

FIG. 12 illustrates a powder X-ray diffraction profile of solid dispersions of compound (1-1) in HPMCAS-MG after 3 month at 40° C. and 75% relative humidity.

FIG. 13A illustrates basal c-MYC gene expression in a panel of acute leukemia cell lines.

FIG. 13B illustrates BRD2/3/4 protein and mRNA expression in a panel of acute leukemia cell lines after exposure to Compound (1-1).

FIG. 13C illustrates c-MYC gene expression in a panel of acute leukemia cell lines after exposure to Compound (1-1).

FIG. 13D illustrates gene expression level of BRD2, BRD3, and BRD4 in a panel of acute leukemia cell lines.

FIG. 13E illustrates the relative level of BRD2, BRD3, and BRD4 mRNA expression in a panel of acute leukemia cell lines after exposure to Compound (1-1).

FIG. 13F illustrates the relative level of HEXIM1 mRNA expression in a panel of acute leukemia cell lines after treatment with Compound (1-1).

FIG. 14A illustrates effect of 500 nM Compound (1-1) for 48 h on the cell cycle in AML cell lines (K562, KG1a, HL60, HEL, NB4, NOMO-1, KG1, OCI-AML3, KASUMI) and ALL cell lines (JURKAT, BV-173, TOM-1, and RS4-11).

FIG. 14B illustrates effect of 500 nM Compound (1-1) for 48 h on the cell cycle in AML cell lines (K562, KG1a, HL60, HEL, NB4, NOMO-1, KG1, OCI-AML3, KASUMI) and ALL cell lines (JURKAT, BV-173, TOM-1, and RS4-11).

FIG. 14C illustrates induction of apoptosis in AML cell lines (HEL, NB4, NOMO-1, OCI-AML-3, KASUMI) and ALL cell lines (JURKAT and RS4-11) patients by exposure to 500 nM Compound (1-1) for 72 h.

FIG. 14D illustrates that 72 h exposure to 500 nM Compound (1-1) activated caspase-3 and induced cytochrome c release, suggesting that BET inhibition leads at least in part to mitochondrial triggered apoptosis.

FIG. 15A illustrates induction of apoptosis in acute leukemia patients by exposure to 500 nM Compound (1-1) for 72 h.

FIG. 15B illustrates induced activation of caspase-3 and mitochondrial cytochrome c release by Compound (1-1) in AML patient samples.

FIG. 15C illustrates c-MYC mRNA expression after treatment with 500 nM Compound (1-1) for 48 h in AML and ALL patient samples.

FIG. 15D illustrates c-MYC, BRD2, and GAPDH protein expression in three AML patient samples after 72 h treatment with 500 nM Compound (1-1).

FIG. 15E illustrates BRD2/3/4 gene expression in AML and ALL patient samples of various subtypes.

FIG. 16A-1 illustrates BRD2/3/4, c-MYC, and GAPDH protein expression in an AML cell line (K562) and an ALL cell line (RS4-11) after exposure to 500 nM Compound (1-1) at 24 h, 48 h, and 72 h.

FIG. 16A-2 illustrates BRD2/3/4, c-MYC, and GAPDH protein expression in AML cell lines (NB4, NOMO-1, and HL60) after exposure to 500 nM Compound (1-1) at 24 h, 48 h, and 72 h.

FIG. 16B-1 illustrates BRD2/3/4, c-MYC, and GAPDH protein expression in AML cell lines (OCI-AML3 and K562) and ALL cell lines (JURKAT, and RS4-11) after exposure to 500 nM JQ1 at 24 h, 48 h, and 72 h.

FIG. 16B-2 illustrates BRD2/3/4, c-MYC, and GAPDH protein expression in AML cell lines (NB4, NOMO-1, and HL60) after exposure to 500 nM JQ1 at 24 h, 48 h, and 72 h.

FIG. 16C illustrates c-MYC gene expression in a panel of AML cell lines (K562, HL60, NB4, KG1, OCI-AML3) and ALL cell lines (JURKAT, RS4-11) after exposure to JQ1.

FIG. 17 illustrates the effect of 25 nM, 100 nM, 250 nM and 500 nM Compound (1-1) for 48 h on the cell cycle in AML cell lines (K562, KG1a, HL60, HEL, NB4, NOMO-1, KG1, OCI-AML3, KASUMI) and ALL cell lines (JURKAT, BV-173, TOM-1, and RS4-11).

FIG. 18A illustrates relative c-MYC mRNA expression in AML cell lines and ALL cell lines as a function of Compound (1-1) induced loss of viability.

FIG. 18B illustrates relative BRD4 mRNA expression in AML cell lines and ALL cell lines as a function of Compound (1-1) induced loss of viability.

FIG. 18C illustrates relative BRD2 mRNA expression in AML cell lines and ALL cell lines as a function of Compound (1-1) induced loss of viability.

FIG. 18D illustrates relative BRD3 mRNA expression in AML cell lines and ALL cell lines as a function of Compound (1-1) induced loss of viability.

FIG. 18E illustrates relative HEXIM1 mRNA expression in AML cell lines and ALL cell lines as a function of Compound (1-1) induced loss of viability.

FIG. 19 illustrates BRD2/3/4 gene expression in AML and ALL patient samples of various subtypes.

FIG. 20A illustrates reduction of cell viability or apoptosis for AML cell lines (K562, HL60, NB4, MONO-1, KG1, OCI-AML3) and ALL cell lines (JURKAT, RS4-11) and cMYC, BRD2/3/4 and HEXIM1 expression levels in cell lines exposed to Compound (1-1).

FIG. 20B illustrates the shade key for FIG. 20A.

### DETAILED DESCRIPTION OF THE INVENTION

The present subject matter will now be described more fully hereinafter with reference to the accompanying Figures and Examples, in which representative embodiments are shown. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entireties.

### I. DEFINITIONS

The term “alkyl group” as used herein refers to a saturated straight or branched hydrocarbon.

The term “substituted alkyl group” refers to an alkyl moiety having one or more substituents replacing hydrogen or one or more carbons of the hydrocarbon backbone.

The term “alkenyl group” whether used alone or as part of a substituent group, for example, “C₁₋₄alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl(2-propenyl), butenyl and the like. Examples include C₁₋₄alkenyl or C₂₋₄alkenyl groups.

The term “C₍ⱼ₋ₖ₎” (where j and k are integers referring to a designated number of carbon atoms) refers to an alkyl, alkenyl, alkynyl, alkoxy or cycloalkyl radical or to the alkyl portion of a radical in which alkyl appears as the prefix root containing from j to k carbon atoms inclusive. For example, C₍₁₋₄₎ denotes a radical containing 1, 2, 3 or 4 carbon atoms.

The terms “halo” or “halogen” as used herein refer to F, Cl, Br, or I.

The term “pharmaceutically acceptable salts” is art-recognized and refers to the relatively non-toxic, inorganic and organic acid addition salts, or inorganic or organic base addition salts of compounds, including, for example, those contained in compositions of the present invention.

The term “solid dispersion” as used herein refers to a group of solid products consisting of at least two different components, generally a hydrophilic carrier and a hydrophobic drug (active ingredient).

The term “chiral” is art-recognized and refers to molecules that have the property of non-superimposability of the mirror image partner, while the term “achiral” refers to molecules which are superimposable on their mirror image partner. A “prochiral molecule” is a molecule that has the potential to be converted to a chiral molecule in a particular process.

The symbol “ [symbol] ” is used to denote a bond that may be a single, a double or a triple bond.

The term “enantiomer” as it is used herein, and structural formulas depicting an enantiomer are meant to include the “pure” enantiomer free from its optical isomer as well as mixtures of the enantiomer and its optical isomer in which the enantiomer is present in an enantiomeric excess, e.g., at least 10%, 25%, 50%, 75%, 90%, 95%, 98%, or 99% enantiomeric excess.

The term “stereoisomers” when used herein consist of all geometric isomers, enantiomers or diastereomers. The present invention encompasses various stereoisomers of these compounds and mixtures thereof. Conformational isomers and rotamers of disclosed compounds are also contemplated.

The term “stereoselective synthesis” as it is used herein denotes a chemical or enzymatic reaction in which a single reactant forms an unequal mixture of stereoisomers during the creation of a new stereocenter or during the transformation of a pre-existing one, and are well known in the art. Stereoselective syntheses encompass both enantioselective and diastereoselective transformations. For examples, see Carreira, E. M. and Kvaerno, L., Classics in Stereoselective Synthesis, WileyVCH: Weinheim, 2009.

The term “spray drying” refers to processes which involve the atomization of the feed suspension or solution into small droplets and rapidly removing solvent from the mixture in a processor chamber where there is a strong driving force for the evaporation (i.e., hot dry gas or partial vacuum or combinations thereof).

As used herein, the term “effective amount” refers to an amount of a thienotriazolodiazepine compound of the present invention or any other pharmaceutically active agent that will elicit a targeted biological or a medical response of a tissue, a biological system, an animal or a human, for instance, intended by a researcher or clinician or a healthcare provider. In some embodiments, the term “effective amount” is used to refer any amount of a thienotriazolodiazepine of the present invention or any other pharmaceutically active agent which is effective at enhancing a normal physiological function.

The term “therapeutically effective amount” as used herein refers to any amount of a thienotriazolodiazepine compound of the present invention or any other pharmaceutically active agent which, as compared to a corresponding patient who has not received such an amount of the thienotriazolodiazepine or the other pharmaceutically active agent, results in improved treatment, healing, prevention, or amelioration of a disease, disorder, or side effect, or a decrease in the rate of advancement of a disease or disorder.

The term “about” means+/−10%. In one embodiment, it means+/−5%.

Throughout this application and in the claims that follow, unless the context requires otherwise, the word “comprise”, or variations such as “comprises” or “comprising”, should be understood to imply the inclusion of a stated integer step or group of integers or steps but not the exclusion of any other integer or step or group of integers or steps. Moreover, the word “comprise” should be understood to imply “consist of”.

### II. METHOD OF USE

The present inventions described herein provide for methods of treating acute myeloid leukemia or acute lymphoid leukemia. The detailed description sets forth the disclosure in various parts: III. Thienotriazolodiazepine Compounds; IV. Formulations; V. Dosage Forms; VI. Dosage; VII. Process; and VIII. Examples. One of skill in the art would understand that each of the various embodiments of methods of treatment include the various embodiments of thienotriazolodiazepine compounds, formulations, dosage forms, dosage and processes described herein.

In some embodiments, the present invention provides a method of treating acute myeloid leukemia or acute lymphoid leukemia in a mammal, administering a pharmaceutically acceptable amount of a thienotriazolodiazepine compound represented by the compound of Formula (1), in particular of Formula (1A), or a pharmaceutically acceptable salt, a solvate, a racemate, an enantiomer, an isomer, or an isotopically-labeled form thereof.

In some embodiments, the present disclosure provides for a method of treating acute myeloid leukemia or acute lymphoid leukemia in a mammal comprising: administering to a patient in need a pharmaceutically acceptable amount of a composition comprising a solid dispersion according to any of the compositions described in Sections III, IV, V and VI described herein.

In some embodiments, the present disclosure provides for methods of treating acute myeloid leukemia or acute lymphoid leukemia in a mammal comprising: administering to a patient in need a pharmaceutically acceptable amount of a composition comprising a pharmaceutical formulation according to any of the compositions described in Sections III, IV, V and VI described herein.

In some embodiments, the present disclosure provides for a compound of Formula (1), in particular of Formula (1A), for use in treating acute myeloid leukemia or acute lymphoid leukemia.

In some embodiments, the present disclosure provides for a solid dispersion according to any of the compositions described in Sections III, IV, V and VI described herein for use in treating acute myeloid leukemia or acute lymphoid leukemia.

In some embodiments, methods of treating acute myeloid leukemia or acute lymphoid leukemia comprise administering a thienotriazolodiazepine compound of the Formula (1)

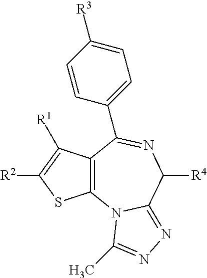

wherein
R¹ is alkyl having a carbon number of 1-4,
R² is a hydrogen atom; a halogen atom; or alkyl having a carbon number of 1-4 optionally substituted by a halogen atom or a hydroxyl group,
R³ is a halogen atom; phenyl optionally substituted by a halogen atom, alkyl having a carbon number of 1-4, alkoxy having a carbon number of 1-4 or cyano; —NR⁵—(CH₂)ₘ—R⁶ wherein R⁵ is a hydrogen atom or alkyl having a carbon number of 1-4, m is an integer of 0-4, and R⁶ is phenyl or pyridyl optionally substituted by a halogen atom; or —NR⁷—CO—(CH₂)ₙ—R⁸ wherein R⁷ is a hydrogen atom or alkyl having a carbon number of 1-4, n is an integer of 0-2, and R⁸ is phenyl or pyridyl optionally substituted by a halogen atom, and
R⁴ is —(CH₂)ₐCO—NH—R⁹ wherein a is an integer of 1-4, and R⁹ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR¹⁰ wherein b is an integer of 1-4, and R¹⁰ is alkyl having a carbon number of 1-4,
including any salts, isomers, enantiomers, racemates, hydrates, solvates, metabolites, and polymorphs thereof.

In some embodiments, Formula (1) is selected from Formula (1A):

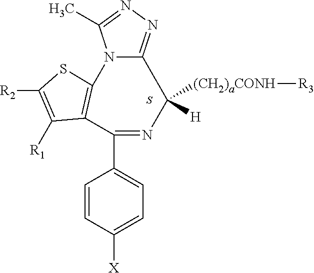

wherein X is a halogen, R¹ is C₁-C₄ alkyl, R² is C₁-C₄ alkyl, a is an integer of 1-4, R³ is C₁-C₄ alkyl, C₁-C₄ hydroxyalkyl, C₁-C₄ alkoxy, phenyl optionally having substituent(s) as defined for R⁹ in Formula (1), or heteroaryl optionally having substituent(s) as defined for R⁹ in Formula (1), a pharmaceutically acceptable salt thereof or a hydrate thereof.

In some embodiments, the present disclosure provides for a compound of Formula (1), in particular a compound of Formula (1A), for use in treating acute myeloid leukemia. In some embodiments, the present disclosure provides for a compound of Formula (1), in particular a compound of Formula (1A), for use in treating acute lymphoid leukemia.

In some embodiments, the thienotriazolodiazepine compound of Formula (1) is formulated as a solid dispersion comprising an amorphous thienotriazolodiazepine compound of Formula (1) or a pharmaceutically acceptable salt thereof or a hydrate thereof; and a pharmaceutically acceptable polymer. Various embodiments of such a solid dispersion are described herein and can be used accordingly.

In some embodiments, the present disclosure provides for a solid dispersion according to any of the compositions described in Sections III, IV, V and VI described herein for use in treating acute myeloid leukemia. In some embodiments, the present disclosure provides for a solid dispersion according to any of the compositions described in Sections III, IV, V and VI described herein for use in treating acute lymphoid leukemia.

In some embodiments of the methods of treating acute myeloid leukemia according to the invention, c-MYC RNA levels are downregulated. In some embodiments of the methods of treating acute myeloid leukemia according to the invention, BRD2, BRD3, and/or BRD4 mRNA levels are upregulated. In other embodiments of the methods of treating acute myeloid leukemia according to the invention, BRD2, BRD3, and/or BRD4 mRNA levels are downregulated. In one embodiment, BRD2, BRD3, and/or BRD4 mRNA levels are downregulated where the AML is resistant to the thienotriazolodiazepine compound being administered. In some embodiments of the methods of treating acute myeloid leukemia according to the invention, HEXIM1 expression is upregulated. In one embodiment, HEXIM1 levels are upregulated where the AML is sensitive to the thienotriazolodiazepine compound being administered.

In some embodiments of the methods of treating acute lymphoid leukemia according to the invention, c-MYC RNA levels are downregulated. In some embodiments of the methods of treating acute lymphoid leukemia according to the invention, BRD2, BRD3, and/or BRD4 mRNA levels are upregulated. In other embodiments of the methods of treating acute lymphoid leukemia according to the invention, BRD2, BRD3, and/or BRD4 mRNA levels are downregulated. In one embodiment, BRD2, BRD3, and/or BRD4 mRNA levels are downregulated where the ALL is resistant to the thienotriazolodiazepine compound being administered. In some embodiments of the methods of treating acute lymphoid leukemia according to the invention, HEXIM1 expression is upregulated. In one embodiment, HEXIM1 levels are upregulated where the ALL is sensitive to the thienotriazolodiazepine compound being administered.

A mammalian subject as used herein can be any mammal. In one embodiment, the mammalian subject includes, but is not limited to, a human; a non-human primate; a rodent such as a mouse, rat, or guinea pig; a domesticated pet such as a cat or dog; a horse, cow, pig, sheep, goat, or rabbit. In one embodiment, the mammalian subject includes, but is not limited to, a bird such as a duck, goose, chicken, or turkey. In one embodiment, the mammalian subject is a human. In one embodiment, the mammalian subject can be either gender and can be any age.

### III. THIENOTRIAZOLODIAZEPINE COMPOUNDS

In one embodiment, the thienotriazolodiazepine compounds, used in the formulations of the present invention, are represented by Formula (1):

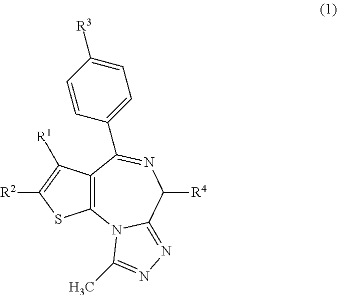

wherein
R¹ is alkyl having a carbon number of 1-4,
R² is a hydrogen atom; a halogen atom; or alkyl having a carbon number of 1-4 optionally substituted by a halogen atom or a hydroxyl group,
R³ is a halogen atom; phenyl optionally substituted by a halogen atom, alkyl having a carbon number of 1-4, alkoxy having a carbon number of 1-4 or cyano; —NR⁵—(CH₂)ₘ—R⁶ wherein R⁵ is a hydrogen atom or alkyl having a carbon number of 1-4, m is an integer of 0-4, and R⁶ is phenyl or pyridyl optionally substituted by a halogen atom; or —NR⁷—CO—(CH₂)ₙ—R⁸ wherein R⁷ is a hydrogen atom or alkyl having a carbon number of 1-4, n is an integer of 0-2, and R⁸ is phenyl or pyridyl optionally substituted by a halogen atom, and
R⁴ is —(CH₂)ₐCO—NH—R⁹ wherein a is an integer of 1-4, and R⁹ is alkyl having a carbon number of 1-4; hydroxyalkyl having a carbon number of 1-4; alkoxy having a carbon number of 1-4; or phenyl or pyridyl optionally substituted by alkyl having a carbon number of 1-4, alkoxy having a carbon number of 1-4, amino or a hydroxyl group or —(CH₂)_{b}—COOR¹⁰ wherein b is an integer of 1-4, and R¹⁰ is alkyl having a carbon number of 1-4,
including any salts, isomers, enantiomers, racemates, hydrates, solvates, metabolites, and polymorphs thereof.

In one embodiment, a suitable alkyl group includes linear or branched alkyl radicals including from 1 carbon atom up to 4 carbon atoms. In one embodiment, a suitable alkyl group includes linear or branched alkyl radicals including from 1 carbon atom up to 3 carbon atoms. In one embodiment, a suitable alkyl group includes linear or branched alkyl radicals include from 1 carbon atom up to 2 carbon atoms. In one embodiment, exemplary alkyl radicals include, but are not limited to, methyl, ethyl, propyl, isopropyl, n-butyl, isobutyl, sec-butyl, tert-butyl. In one embodiment, exemplary alkyl groups include, but are not limited to, methyl, ethyl, propyl, isopropyl, 2-methyl-1-propyl, and 2-methyl-2-propyl.

In some embodiments, the present invention provides pharmaceutically acceptable salts, solvates, including hydrates, and isotopically-labeled forms of the thienotriazolodiazepine compounds described herein. In one embodiment, pharmaceutically acceptable salts of the thienotriazolodiazepine compounds include acid addition salts formed with inorganic acids. In one embodiment, pharmaceutically acceptable inorganic acid addition salts of the thienotriazolodiazepine include salts of hydrochloric, hydrobromic, hydroiodic, phosphoric, metaphosphoric, nitric and sulfuric acids. In one embodiment, pharmaceutically acceptable salts of the thienotriazolodiazepine compounds include acid addition salts formed with organic acids. In one embodiment, pharmaceutically acceptable organic acid addition salts of the thienotriazolodiazepine include salts of tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and 4-methyl benzenesulfonic acids.

The present invention provides pharmaceutically acceptable isotopically-labeled forms of the thienotriazolodiazepine compounds, described herein, wherein one or more atoms are replaced by atoms having the same atomic number, but an atomic mass or mass number different from the atomic mass or mass number usually found in nature. Examples of isotopes suitable for inclusion in the thienotriazolodiazepine compounds include isotopes of hydrogen, e.g., ²H and ³H, carbon, e.g., ¹¹C, ¹³C and ¹⁴C, chlorine, e.g., ³⁶Cl, fluorine, e.g., ¹⁸F, iodine, e.g., ¹²³I and ¹²⁵I, nitrogen, e.g., ¹³N and ¹⁵N, oxygen, e.g., ¹⁵O, ¹⁷O and ¹⁸O, and sulfur, e.g., ³⁵S. Isotopically-labeled forms of the thienotriazolodiazepine compounds generally can be prepared by conventional techniques known to those skilled in the art.

Certain isotopically-labeled forms of the compound of Formula (1), for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be used in Positron Emission Tomography (PET) studies for examining substrate receptor occupancy.

In some embodiments, the thienotriazolodiazepine compounds disclosed herein can exist in solvated as well as unsolvated forms with pharmaceutically acceptable solvents. It will be understood by those skilled-in the art that a solvate is a complex of variable stoichiometry formed by a solute (in this case, the thienotriazolodiazepine compounds described herein) and a solvent. It is preferred that such solvents not interfere with the biological activity of the solute (the thienotriazolodiazepine compounds). Examples of suitable solvents for solvate formation include, but are not limited to, water, methanol, dimethyl sulfoxide, ethanol and acetic acid. Suitably the solvent used is a pharmaceutically acceptable solvent. Suitably the solvent used is water. In one embodiment, pharmaceutically acceptable solvates of the thienotriazolodiazepine compounds, described herein, include ethanol solvate, a isopropanol solvate, a dioxolane solvate, a tetrahydrofuran solvate, a dimethyl sulfoxide solvate, tert-butanol solvate, 2-butanol solvate, dioxolane solvate, 1,3-Dimethyl-3,4,5,6-tetrahydro-2(1H)-pyrimidinone (“DMPU”) solvate, 1,3-dimethylimidazolidinone (“DMI”) solvate, and 1,3-dimethylimidazolidinone (“DMP”) solvate, or mixtures thereof.

In some embodiments, the thienotriazolodiazepine compounds, described herein, may contain one or more chiral centers and/or double bonds and, therefore, may exist as geometric isomers, enantiomers or diastereomers. The enantiomer and diastereomers of the thienotriazolodiazepine compounds may be designated in accordance with the Cahn-Ingold-Prelog convention, which assigns an “R” or “S” descriptor to each stereocenter (also sometimes referred to as a chiral center) and an E or Z descriptor to each carbon-carbon double bond (to designate geometric isomers) so that the configuration of the entire molecule can be specified uniquely by including the descriptors in its systematic name.

In some embodiments, the thienotriazolodiazepine compounds, described herein, may exist as a racemic mixture, or racemate, which includes equal amounts of left- and right-handed enantiomers of a chiral molecule. Such a racemic mixture may be denoted by the prefix (±)- or dl-, indicating an equal (1:1) mixture of dextro and levo isomers. Also, the prefix rac- (or racem-) or the symbols RS and SR may be used to designate the racemic mixture.

Geometric isomers, resulting from the arrangement of substituents around a carbon-carbon double bond or arrangement of substituents around a cycloalkyl or heterocyclic ring, can also exist in the compounds of the present invention. In some embodiments, the symbol -̅-̅-̅-̅-̅-̅ may be used to denote a bond that may be a single, double or triple bond. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of a plane of a ring are designated “cis/trans” or “Z/E.”

In some embodiments, thienotriazolodiazepine compounds disclosed herein may exist in single or multiple crystalline forms or polymorphs. In one embodiment, a thienotriazolodiazepine compound disclosed herein comprises an amorphous form thereof. In one embodiment, a thienotriazolodiazepine compound disclosed herein comprises a single polymorph thereof. In another embodiment, a thienotriazolodiazepine compound disclosed herein comprises a mixture of polymorphs thereof. In another embodiment, the compound is in a crystalline form.

In some embodiments, thienotriazolodiazepine compounds disclosed herein may exist as a single enantiomer or in enatiomerically enriched forms. In one embodiment, a thienotriazolodiazepine compound disclosed herein exists in an enantiomeric excess of more than 80%. In one embodiment, a thienotriazolodiazepine compound disclosed herein exists in an enantiomeric excess of more than 90%. In one embodiment, a thienotriazolodiazepine compound disclosed herein exists in an enantiomeric excess of more than 98%. In one embodiment, a thienotriazolodiazepine compound disclosed herein exists in an enantiomeric excess of more than 99%. In some embodiments, a thienotriazolodiazepine compound disclosed herein exists in an enantiomeric excess selected from the group consisting of at least 10%, at least 25%, at least 50%, at least 75%, at least 90%, at least 95%, at least 98%, at least and at least 99% enantiomeric excess.

For a pair of enantiomers, enantiomeric excess (ee) of enantiomer E1 in relation to enantiomer E2 can be calculated using the following equation eq. (1):

$$\% enantiomeric excess of E 1 = \frac{( E 1 - E 2 ) \times 100 \%}{( E 1 + E 2 )} \qquad eq . ( 1 )$$

Relative amounts of E1 and E2 can be determined by chiral high performance liquid chromatography (HPLC), nuclear magnetic resonance (NMR) or any other suitable methods. In some embodiments, purity of an enantiomeric compound may refer to the amount of the enantiomers E1 and E2, relative to the amount of other materials, which may notably include byproducts and/or unreacted reactants or reagents.

Representative thienotriazolodiazepine compounds of Formula (1) include, but are not limited to, the thienotriazolodiazepine compounds (1-1) to (1-18), which are listed in the following Table A. Compound (1-1) of Table A will also be referred to herein as OTX-015, OTX015 or Y803.

TABLE A
Exemplary compounds used in the invention:

| | |
|---|---|
| 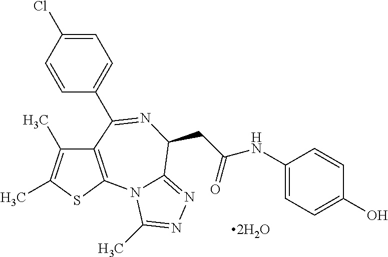 [SMILES: Cc1sc2c(c1C)C(c1ccc(Cl)cc1)=N[C@@H](CC(=O)Nc1ccc(O)cc1)c1nnc(C)n1-2] | (1-1) |
| 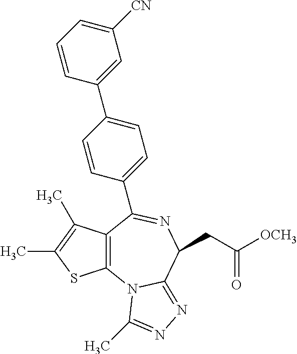 [SMILES: COC(=O)C[C@@H]1N=C(c2ccc(-c3cccc(C#N)c3)cc2)c2c(sc(C)c2C)-n2c(C)nnc21] | (1-2) |
| 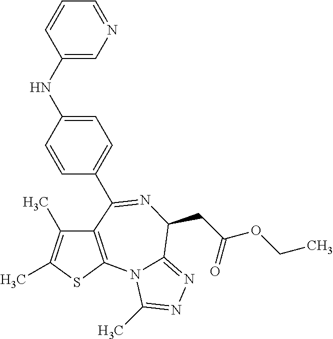 [SMILES: CCOC(=O)C[C@@H]1N=C(c2ccc(Nc3cccnc3)cc2)c2c(sc(C)c2C)-n2c(C)nnc21] | (1-3) |
| 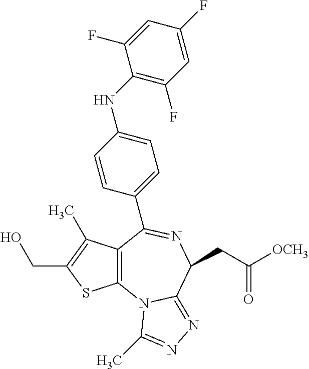 [SMILES: COC(=O)C[C@@H]1N=C(c2ccc(Nc3c(F)cc(F)cc3F)cc2)c2c(sc(CO)c2C)-n2c(C)nnc21] | (1-4) |
| 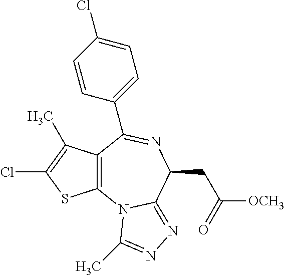 [SMILES: COC(=O)C[C@@H]1N=C(c2ccc(Cl)cc2)c2c(sc(Cl)c2C)-n2c(C)nnc21] | (1-5) |
| 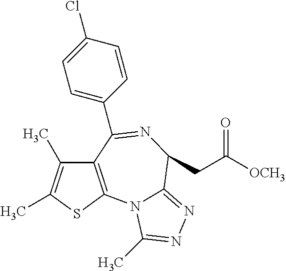 [SMILES: COC(=O)C[C@@H]1N=C(c2ccc(Cl)cc2)c2c(sc(C)c2C)-n2c(C)nnc21] | (1-6) |
| 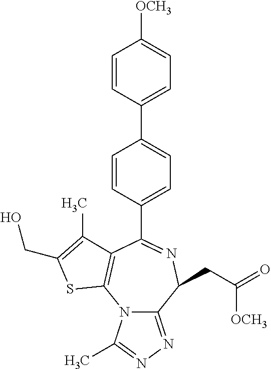 [SMILES: COC(=O)C[C@@H]1N=C(c2ccc(-c3ccc(OC)cc3)cc2)c2c(sc(CO)c2C)-n2c(C)nnc21] | (1-7) |
| 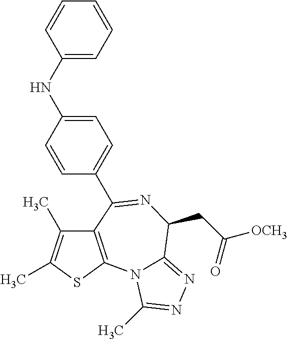 [SMILES: COC(=O)C[C@@H]1N=C(c2ccc(Nc3ccccc3)cc2)c2c(sc(C)c2C)-n2c(C)nnc21] | (1-8) |
| 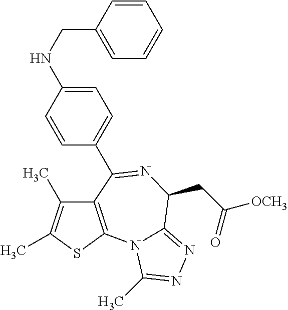 [SMILES: COC(=O)C[C@@H]1N=C(c2ccc(NCc3ccccc3)cc2)c2c(sc(C)c2C)-n2c(C)nnc21] | (1-9) |
| 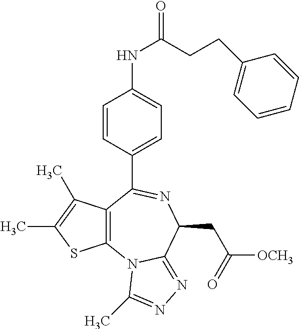 [SMILES: COC(=O)C[C@@H]1N=C(c2ccc(NC(=O)CCc3ccccc3)cc2)c2c(sc(C)c2C)-n2c(C)nnc21] | (1-10) |
| 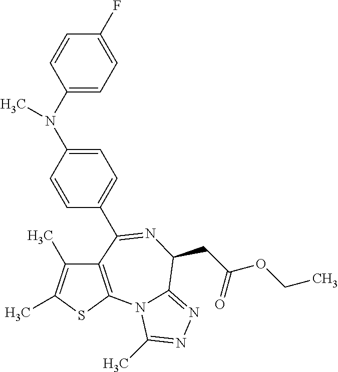 [SMILES: CCOC(=O)C[C@@H]1N=C(c2ccc(N(C)c3ccc(F)cc3)cc2)c2c(sc(C)c2C)-n2c(C)nnc21] | (1-11) |
| 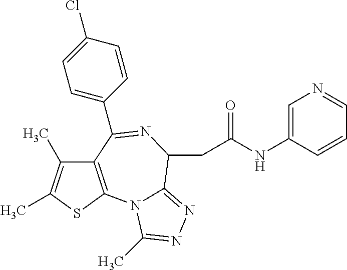 [SMILES: Cc1sc2c(c1C)C(c1ccc(Cl)cc1)=NC(CC(=O)Nc1cccnc1)c1nnc(C)n1-2] | (1-12) |
| 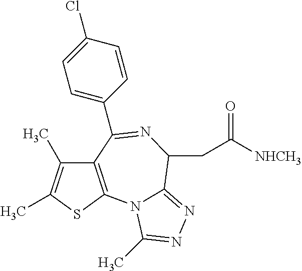 [SMILES: CNC(=O)CC1N=C(c2ccc(Cl)cc2)c2c(sc(C)c2C)-n2c(C)nnc21] | (1-13) |
| 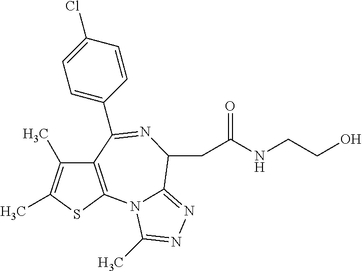 [SMILES: Cc1sc2c(c1C)C(c1ccc(Cl)cc1)=NC(CC(=O)NCCO)c1nnc(C)n1-2] | (1-14) |
| 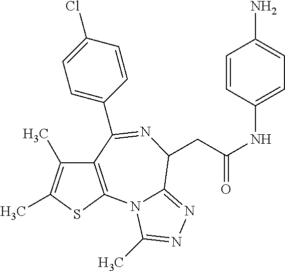 [SMILES: Cc1sc2c(c1C)C(c1ccc(Cl)cc1)=NC(CC(=O)Nc1ccc(N)cc1)c1nnc(C)n1-2] | (1-15) |
| 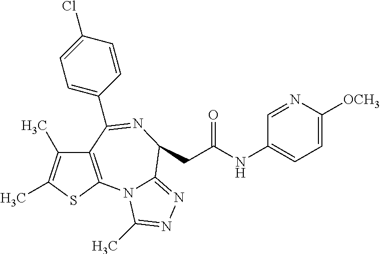 [SMILES: COc1ccc(NC(=O)C[C@@H]2N=C(c3ccc(Cl)cc3)c3c(sc(C)c3C)-n3c(C)nnc32)cn1] | (1-16) |
| 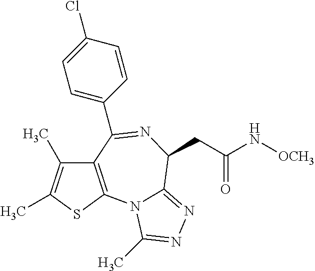 [SMILES: CONC(=O)C[C@@H]1N=C(c2ccc(Cl)cc2)c2c(sc(C)c2C)-n2c(C)nnc21] | (1-17) |
| 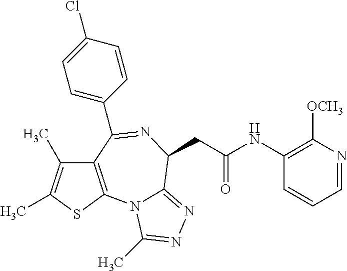 [SMILES: COc1ncccc1NC(=O)C[C@@H]1N=C(c2ccc(Cl)cc2)c2c(sc(C)c2C)-n2c(C)nnc21] | (1-18) |

In some embodiments, thienotriazolodiazepine compounds of Formula (1) include (i) (S)-2-[4-(4-chlorophenyl)-2,3,9-trimethyl-6H-thieno[3,2-f][1,2,4]triazolo[4,3-a][1,4]diazepin-6-yl]-N-(4-hydroxyphenyl)acetamide or a dihydrate thereof, (ii) methyl (S)-{4-(3′-cyanobiphenyl-4-yl)-2,3,9-trimethyl-6H-thieno[3,2-f][1,2,4]tri-azolo[4,3-a][1,4]diazepin-6-yl} acetate, (iii) methyl (S){2,3,9-trimethyl-4-(4-phenylaminophenyl)-6H-thieno[3,2-f][1,2,4]triazolo[4,3-a][1,4]diazepin-6-yl} acetate; and (iv) methyl (S)-{2,3,9-trimethyl-4-[4-(3-phenylpropionylamino)phenyl]-6H-thieno[3,2-f][1,2,4]triazolo[4,3-a][1,4]diazepin-6-yl} acetate.

In some embodiments, thienotriazolodiazepine compounds of Formula (1) include (S)-2-[4-(4-chlorophenyl)-2,3,9-trimethyl-6H-thieno[3,2-f][1,2,4]triazolo[4,3-a][1,4]diazepin-6-yl]-N-(4-hydroxyphenyl)acetamide dihydrate.

In some embodiments, thienotriazolodiazepine compounds of Formula (1) include (S)-2-[4-(4-chlorophenyl)-2,3,9-trimethyl-6H-thieno[3,2-f][1,2,4]triazolo[4,3-a][1,4]diazepin-6-yl]-N-(4-hydroxyphenyl)acetamide.

### IV. FORMULATIONS

The compound of Formula (1) presents highly specific difficulties in relation to administration generally and the preparation of galenic compositions in particular, including the particular problems of drug bioavailability and variability in inter- and intra-patient dose response, necessitating development of a non-conventional dosage form with respect to the practically water-insoluble properties of the compound.

Previously, it had been found that the compound of Formula (1) could be formulated as a solid dispersion with the carrier ethyl acrylate-methyl methacrylate-trimethylammonioethyl methacrylate chloride copolymer (Eudragit RS, manufactured by Rohm) to provide an oral formulation that preferentially released the pharmaceutical ingredient in the lower intestine for treatment of inflammatory bowel diseases such as ulcerative colitis and Crohn's disease (US Patent Application 20090012064 A1, published Jan. 8, 2009). It was found, through various experiments, including animal tests, that in inflammatory bowel diseases drug release in a lesion and a direct action thereof on the inflammatory lesion were more important than the absorption of the drug into circulation from the gastrointestinal tract.

It has now been unexpectedly found that thienotriazolodiazepine compounds, according to Formula (1), pharmaceutically acceptable salts, solvates, including hydrates, racemates, enantiomers isomers, and isotopically-labeled forms thereof, can be formulated as a solid dispersion with pharmaceutically acceptable polymers to provide an oral formulation that provides high absorption of the pharmaceutical ingredient into the circulation from the gastrointestinal tract for treatment of diseases other than inflammatory bowel diseases. Studies in both dogs and humans have confirmed high oral bioavailability of these solid dispersions compared with the Eudragit solid dispersion formulation previously developed for the treatment of inflammatory bowel disease.

Solid dispersions are a strategy to improve the oral bioavailability of poorly water soluble drugs.

The term “solid dispersion” as used herein refers to a group of solid products including at least two different components, generally a hydrophilic carrier and a hydrophobic drug, the thienotriazolodiazepine compounds, according to Formula (1). Based on the drug's molecular arrangement within the dispersion, six different types of solid dispersions can be distinguished. Commonly, solid dispersions are classified as simple eutectic mixtures, solid solutions, glass solution and suspension, and amorphous precipitations in a crystalline carrier. Moreover, certain combinations can be encountered, for example, in the same sample some molecules may be present in clusters while some are molecularly dispersed.

In one embodiment, the thienotriazolodiazepine compounds, according to Formula (1) can be dispersed molecularly, in amorphous particles (clusters). In another embodiment, the thienotriazolodiazepine compounds, according to Formula (1) can be dispersed as crystalline particles. In one embodiment, the carrier can be crystalline. In another embodiment, the carrier can be amorphous.

In one embodiment, the present invention provides a pharmaceutical composition comprising a solid dispersion of a thienotriazolodiazepine compound, in accordance with Formula (1), or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is hypromellose acetate succinate (also called hydroxypropylmethylcellulose acetate succinate or HPMCAS). In one embodiment, the dispersion has a thienotriazolodiazepine compound to hydroxypropylmethylcellulose acetate succinate (HPMCAS) weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1). In some embodiments, the hydroxypropylmethyl cellulose acetate succinates (HPMCAS), may include M grade having 9% acetyl/11% succinoyl (e.g., HPMCAS having a mean particle size of 5 μm (i.e., HPMCAS-MF, fine powder grade) or having a mean particle size of 1 mm (i.e., HPMCAS-MG, granular grade)), H grade having 12% acetyl/6% succinoyl (e.g., HPMCAS having a mean particle size of 5 μm (i.e., HPMCAS-HF, fine powder grade) or having a mean particle size of 1 mm (i.e., HPMCAS-HG, granular grade)), and L grade having 8% acetyl/15% succinoyl (e.g., HPMCAS having a mean particle size of 5 μm (i.e., HPMCAS-LF, fine powder grade) or having a mean particle size of 1 mm (i.e., HPMCAS-LG, granular grade).

In one embodiment, the present invention provides a pharmaceutical composition comprising a solid dispersion of a thienotriazolodiazepine compound of Formula (1) or a pharmaceutically acceptable salt, a solvate, including a hydrate, a racemate, an enantiomer, an isomer, or an isotopically-labeled form thereof in a pharmaceutically acceptable polymer. In one embodiment, the pharmaceutically acceptable polymer is polyvinylpyrrolidone (also called povidone or PVP). In one embodiment, the dispersion has a thienotriazolodiazepine compound to PVP weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to about 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1). In some embodiments, the polyvinyl pyrrolidones may have molecular weights of about 2,500 (Kollidon®12 PF, weight-average molecular weight between 2,000 to 3,000), about 9,000 (Kollidon® 17 PF, weight-average molecular weight between 7,000 to 11,000), about 25,000 (Kollidon® 25, weight-average molecular weight between 28,000 to 34,000), about 50,000 (Kollidon® 30, weight-average molecular weight between 44,000 to 54,000), and about 1,250,000 (Kollidon® 90 or Kollidon® 90F, weight-average molecular weight between 1,000,000 to 1,500,000).

In one embodiment, a pharmaceutical composition of the present invention comprises a solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to about 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1.

In one embodiment, a pharmaceutical composition of the present invention comprises a solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1.

In some embodiments, a pharmaceutical composition comprising a solid dispersion is prepared by spray drying.

In one embodiment, a pharmaceutical composition of the present invention comprises a spray dried solid dispersion of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of compound (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of compound (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 130° C. to 140° C. In some such embodiments, the solid dispersion was exposed to a relative humidity of 75% at 40° C. for at least one month. In other such embodiments, the single Tg occurs at about 135° C. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of an amorphous form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In some embodiments, the solid dispersion exhibits a single inflection for the glass transition temperature (Tg). In some embodiments, the single Tg occurs between 175° C. to 185° C. In some such embodiments, the solid dispersion was exposed to a relative humidity of 75% at 40° C. for at least one month. In other such embodiments, the single Tg occurs at about 179° C. In some embodiments, the solid dispersion exhibits an X-ray powder diffraction pattern substantially free of diffraction lines associated with crystalline thienotriazolodiazepine compound of Formula (1). For the purpose of this application “substantially free” shall mean the absence of a diffraction line, above the amorphous halo, at about 21° 2-theta associated with crystalline thienotriazolodiazepine compound of Formula (1).

In one embodiment, a pharmaceutical composition of the present invention comprises a spray dried solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is hypromellose acetate succinate. In one embodiment, the weight ratio of thienotriazolodiazepine compound of Formula (1) to hypromellose acetate succinate ranges from 1:3 to 1:1.

In one embodiment, a pharmaceutical composition of the present invention comprises a spray dried solid dispersion of a crystalline form of a thienotriazolodiazepine compound of Formula (1) or a pharmaceutically acceptable salt, a solvate, including a hydrate, a racemate, an enantiomer, an isomer, or an isotopically-labeled form thereof and a pharmaceutically acceptable polymer. In one embodiment, the pharmaceutically acceptable polymer is polyvinylpyrrolidone. In one embodiment, the weight ratio of thienotriazolodiazepine compound of Formula (1) to polyvinylpyrrolidone ranges from 1:3 to 1:1.

In one preferred embodiment, the present invention provides a pharmaceutical composition comprising a solid dispersion of 2-[(6S)-4-(4-chlorophenyl)-2,3,9-trimethyl-6H-thienol[3,2-f]-[1,2,4]triazolo[4,3-a][1,4]diazepin-6-yl]-N-(4-hydroxyphenyl)acetamide dihydrate, compound (1-1):

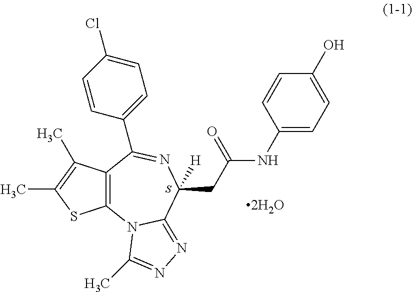

or a pharmaceutically acceptable salt, a solvate, including a hydrate, a racemate, an enantiomer, an isomer, or an isotopically-labeled form and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In another embodiment, the pharmaceutical composition comprises a solid dispersion compound (1-1) or a pharmaceutically acceptable salt, a solvate, including a hydrate, a racemate, an enantiomer, an isomer, or an isotopically-labeled form; and a pharmaceutically acceptable polymer. In one embodiment, the pharmaceutically acceptable polymer is PVP. In one embodiment, the dispersion has compound (1-1) and PVP in a weight ratio 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 175° C. to 185° C. In other such embodiments, the single Tg occurs at about 179° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of an amorphous form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 130° C. to 140° C. In other such embodiments, the single Tg occurs at about 135° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of an amorphous form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is PVP. In one embodiment, the dispersion has compound (1-1) and PVP in a weight ratio 1:3 to 1:1. In one embodiment, at least some portion of the thienotriazolodiazepine compound is homogeneously dispersed throughout the solid dispersion. In another embodiment, the thienotriazolodiazepine compound is homogeneously dispersed throughout the solid dispersion. In one embodiment, the solid dispersion is spray dried. In some embodiments, the solid dispersion exhibits a single inflection for the glass transition temperature (Tg). In some embodiments, the single Tg occurs between 175° C. to 185° C. In other such embodiments, the single Tg occurs at about 189° C. In some such embodiments, the solid dispersion was exposed to a relative humidity of 75% at 40° C. for at least one month. In some embodiments, the solid dispersion exhibits an X-ray powder diffraction pattern substantially free of diffraction lines associated with crystalline thienotriazolodiazepine compound (1-1). For the purpose of this application “substantially free” shall mean the absence of a diffraction line, above the amorphous halo, at about 21° 2-theta associated with crystalline thienotriazolodiazepine compound (1-1).

In one embodiment, a pharmaceutical composition of the present invention comprises a solid dispersion of a crystalline form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is HPMCAS. In one embodiment, the dispersion has compound (1-1) and HPMCAS in a weight ratio of 1:3 to 1:1. In one embodiment, the solid dispersion is spray dried.

In one embodiment, a pharmaceutical composition of the present invention comprises a solid dispersion of a crystalline form of a thienotriazolodiazepine compound (1-1) or a pharmaceutically acceptable salt, a solvate, including a hydrate, a racemate, an enantiomer, an isomer, or an isotopically-labeled form thereof; and a pharmaceutically acceptable polymer. In one embodiment, the pharmaceutically acceptable polymer is PVP. In one embodiment, the dispersion has compound (1-1) and PVP in a weight ratio 1:3 to 1:1. In one embodiment, the solid dispersion is spray dried.

The solid dispersions of the invention, described herein, exhibit especially advantageous properties when administered orally. Examples of advantageous properties of the solid dispersions include, but are not limited to, consistent and high level of bioavailability when administered in standard bioavailability trials in animals or humans. The solid dispersions of the invention can include a solid dispersion comprising thienotriazolodiazepine compound of Formula (1) and a polymer and additives. In some embodiments, the solid dispersions can achieve absorption of the thienotriazolodiazepine compound of Formula (1) into the bloodstream that cannot be obtained by merely admixing the thienotriazolodiazepine compound of Formula (1) with additives since the thienotriazolodiazepine compound of Formula (1) drug has negligible solubility in water and most aqueous media. The bioavailability, of thienotriazolodiazepine compound of Formula (1) or of thienotriazolodiazepine compound (1-1) may be measured using a variety of in vitro and/or in vivo studies. The in vivo studies may be performed, for example, using rats, dogs or humans.

The bioavailability may be measured by the area under the curve (AUC) value obtained by plotting a serum or plasma concentration, of the thienotriazolodiazepine compound of Formula (1) or thienotriazolodiazepine compound (1-1), along the ordinate (Y-axis) against time along the abscissa (X-axis). The AUC value of the thienotriazolodiazepine compound of Formula (1) or thienotriazolodiazepine compound (1-1) from the solid dispersion, is then compared to the AUC value of an equivalent concentration of crystalline thienotriazolodiazepine compound of Formula (1) or crystalline thienotriazolodiazepine compound (1-1) without polymer. In some embodiments, the solid dispersion provides an area under the curve (AUC) value, when administered orally to a dog, that is selected from: at least 0.4 times, 0.5 times, 0.6 time, 0.8 time, 1.0 times, a corresponding AUC value provided by a control composition administered intravenously to a dog, wherein the control composition comprises an equivalent quantity of a crystalline thienotriazolodiazepine compound of Formula I.

The bioavailability may be measured by in vitro tests simulating the pH values of a gastric environment and an intestine environment. The measurements may be made by suspending a solid dispersion of the thienotriazolodiazepine compound of Formula (1) or thienotriazolodiazepine compound (1-1), in an aqueous in vitro test medium having a pH between 1.0 to 2.0, and the pH is then adjusted to a pH between 5.0 and 7.0, in a control in vitro test medium. The concentration of the amorphous thienotriazolodiazepine compound of Formula (1) or amorphous thienotriazolodiazepine compound (1-1) may be measured at any time during the first two hours following the pH adjustment. In some embodiments, the solid dispersion provides a concentration, of the amorphous thienotriazolodiazepine compound of Formula (1) or amorphous thienotriazolodiazepine compound (1-1), in an aqueous in vitro test medium at pH between 5.0 to 7.0 that is selected from: at least 5-fold greater, at least 6 fold greater, at least 7 fold greater, at least 8 fold greater, at least 9 fold greater or at least 10 fold greater, compared to a concentration of a crystalline thienotriazolodiazepine compound of Formula (1) or crystalline thienotriazolodiazepine compound (1-1), without polymer.

In other embodiments, the concentration of the amorphous thienotriazolodiazepine compound of Formula (1) or amorphous thienotriazolodiazepine compound (1-1), from the solid dispersion placed in an aqueous in vitro test medium having a pH of 1.0 to 2.0, is: at least 40%, at least 50% higher, at least 60%, at least 70%; at least 80%, than a concentration of a crystalline thienotriazolodiazepine compound of Formula (1) without polymer. In some such embodiments, the polymer of the solid dispersion is HPMCAS. In some such embodiments, the polymer of the solid dispersion is PVP.

In other embodiments, a concentration of the amorphous thienotriazolodiazepine compound of Formula (1) or amorphous thienotriazolodiazepine compound (1-1), from the solid dispersion, is: at least 40%, at least 50% higher, at least 60%, at least 70%; at least 80%, compared to a concentration of thienotriazolodiazepine compound of Formula (1), from a solid dispersion of thienotriazolodiazepine compound of the Formula (1) and a pharmaceutically acceptable polymer selected from the group consisting of: hypromellose phthalate and ethyl acrylate-methyl methacrylate-trimethylammonioethyl methacrylate chloride copolymer, wherein each solid dispersion was placed in an aqueous in vitro test medium having a pH of 1.0 to 2.0. In some such embodiments, the polymer of the solid dispersion is HPMCAS. In some such embodiments, the polymer of the solid dispersion is PVP.

In some embodiments, the solid dispersions, described herein, exhibit stability against recrystallization of the thienotriazolodiazepine compound of the Formula (1) or the thienotriazolodiazepine compound (1-1) when exposed to humidity and temperature over time. In one embodiment, the concentration of the amorphous thienotriazolodiazepine compound of the Formula (1) or the thienotriazolodiazepine compound (1-1) which remains amorphous is selected from: at least 90%, at least 91%, at least 92%, at least 93%, at least 94%, at least 95%, at least 96%, at least 97%, at least 98% and at least 99%.

### V. DOSAGE FORMS

Suitable dosage forms that can be used with the solid dispersions of the present invention include, but are not limited to, capsules, tablets, mini-tablets, beads, beadlets, pellets, granules, granulates, and powder. Suitable dosage forms may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxypropylmethylcellulose acetate succinate (HPMCAS). In some embodiments, certain combinations can be encountered, for example, in the same sample some molecules of the thienotriazolodiazepine of the present invention may be present in clusters while some are molecularly dispersed with a carrier.

In some embodiments, the solid dispersions of the invention may be formulated as tablets, caplets, or capsules. In one some embodiments, the solid dispersions of the invention may be formulated as mini-tablets or pour-into-mouth granules, or oral powders for constitution. In some embodiments, the solid dispersions of the invention are dispersed in a suitable diluent in combination with other excipients (e.g., re-crystallization/precipitation inhibiting polymers, tast-emasking components, etc) to give a ready-to-use suspension formulation. In some embodiments, the solid dispersions of the invention may be formulated for pediatric treatment.

In one embodiment, the pharmaceutical composition of the present invention is formulated for oral administration. In one embodiment, the pharmaceutical composition comprises a solid dispersion, according to the various embodiments described herein, comprising a thienotriazolodiazepine compound of Formula (1) or a pharmaceutically acceptable salt, a solvate, including a hydrate, a racemate, an enantiomer, an isomer, or an isotopically-labeled form thereof; and a polymer carrier. In one embodiment, the pharmaceutical composition further includes one or more additives such as disintegrants, lubricants, glidants, binders, and fillers.

Examples of suitable pharmaceutically acceptable lubricants and pharmaceutically acceptable glidants for use with the pharmaceutical composition include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose, glyceryl behenate, stearic acid, hydrogenated castor oil, glyceryl monostearate, and sodium stearyl fumarate.

Examples of suitable pharmaceutically acceptable binders for use with the pharmaceutical composition include, but are not limited to starches; celluloses and derivatives thereof, e.g., microcrystalline cellulose (e.g., AVICEL PH from FMC), hydroxypropyl cellulose, hydroxyethyl cellulose, and hydroxypropylmethylcellulose (HPMC, e.g., METHOCEL from Dow Chemical); sucrose, dextrose, corn syrup; polysaccharides; and gelatin.

Examples of suitable pharmaceutically acceptable fillers and pharmaceutically acceptable diluents for use with the pharmaceutical composition include, but are not limited to, confectioner's sugar, compressible sugar, dextrates, dextrin, dextrose, lactose, mannitol, microcrystalline cellulose (MCC), powdered cellulose, sorbitol, sucrose, and talc.

In some embodiments, excipients may serve more than one function in the pharmaceutical composition. For example, fillers or binders may also be disintegrants, glidants, anti-adherents, lubricants, sweeteners and the like.

In some embodiments, the pharmaceutical compositions of the present invention may further include additives or ingredients, such as antioxidants (e.g., ascorbyl palmitate, butylated hydroxylanisole (BHA), butylated hydroxytoluene (BHT), α-tocopherols, propyl gallate, and fumaric acid), antimicrobial agents, enzyme inhibitors, stabilizers (e.g., malonic acid), and/or preserving agents.

Generally, the pharmaceutical compositions of the present invention may be formulated into any suitable solid dosage form. In some embodiments, the solid dispersions of the invention are compounded in unit dosage form, e.g., as a capsule, or tablet, or a multi-particulate system such as granules or granulates or a powder, for administration.

In one embodiment, a pharmaceutical compositions includes a solid dispersion of a thienotriazolodiazepine compound of Formula (1), according to the various embodiments of solid dispersions described herein, and hydroxypropylmethylcellulose acetate succinate (HPMCAS), wherein the thienotriazolodiazepine compound is amorphous in the solid dispersion and has a thienotriazolodiazepine compound to hydroxypropylmethylcellulose acetate succinate (HPMCAS), weight ratio of 1:3 to 1:1; 45-50 wt. % of lactose monohydrate; 35-40 wt. % of microcrystalline cellulose; 4-6 wt. % of croscarmellose sodium; 0.8-1.5 wt. % of colloidal silicon dioxide; and 0.8-1.5 wt. % of magnesium stearate.

### VI. DOSAGE

In one embodiment, the present invention provides a pharmaceutical composition that may be formulated into any suitable solid dosage form. In one embodiment, a pharmaceutical composition in accordance with the present invention comprises one or more of the various embodiments of the thienotriazolodiazepine of Formula (1) as described herein in a dosage amount ranging from about 10 mg to about 100 mg. In one embodiment, the pharmaceutical composition of the present invention includes one or more of the various embodiments of the thienotriazolodiazepine of Formula (1) as described herein in a dosage amount selected from the group consisting of from about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, and about 10 mg to about 20 mg. In one embodiment, the pharmaceutical composition of the present invention includes one or more of the various embodiments of the thienotriazolodiazepine of Formula (1) as described herein in a dosage amount selected from the group consisting of about 10 mg, about 50 mg, about 75 mg, about 100 mg.

In some embodiments, the methods of the present invention includes administering to a subject in need thereof one or more of the various embodiments of the thienotriazolodiazepine of Formula (I) as described herein in a dosage amount selected from the group consisting of about 1 mg, about 2 mg, about 2.5 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, and about 150 mg, and in a dosage form selected from the group consisting of once weekly, once daily every sixth day, once daily every fifth day, once daily every fourth day, once daily every third day, once daily every other day, once daily, twice daily, three times daily, four times daily, and five times daily. In another embodiment, any of the foregoing dosage amounts or dosage forms is decreased periodically or increased periodically.

In some embodiments, the methods of the present invention include administering to a subject in need thereof a thienotriazolodiazepine selected from the group consisting of compounds (1-1), (1-2), (1-3), (1-4), (1-5), (1-6), (1-7), (1-8), (1-9), (1-10), (1-11), (1- 12), (1-13), (1-14), (1-15), (1-16), (1-17), and (1-18), in a dosage amount selected from the group consisting of about 1 mg, about 2 mg, about 2.5 mg, about 3 mg, about 4 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, and about 150 mg, and in a dosage form selected from the group consisting of once weekly, once daily every sixth day, once daily every fifth day, once daily every fourth day, once daily every third day, once daily every other day, once daily, twice daily, three times daily, four times daily, and five times daily. In another embodiment, any of the foregoing dosage amounts or dosage forms is decreased periodically or increased periodically.

Such unit dosage forms are suitable for administration 1 to 5 times daily depending on the particular purpose of therapy, the phase of therapy, and the like. In one embodiment, the dosage form may be administered to a subject in need thereof at least once daily for at least two successive days. In one embodiment, the dosage form may be administered to a subject in need thereof at least once daily on alternative days. In one embodiment, the dosage form may be administered to a subject in need thereof at least weekly and divided into equal and/or unequal doses. In one embodiment, the dosage form may be administered to a subject in need thereof weekly, given either on three alternate days and/or 6 times per week. In one embodiment, the dosage form may be administered to a subject in need thereof in divided doses on alternate days, every third day, every fourth day, every fifth day, every sixth day and/or weekly. In one embodiment, the dosage form may be administered to a subject in need thereof two or more equally or unequally divided doses per month.

The dosage form used, e.g., in a capsule, tablet, mini-tablet, beads, beadlets, pellets, granules, granulates, or powder may be coated, for example using an enteric coating. Suitable coatings may comprise but are not limited to cellulose acetate phthalate, hydroxypropylmethylcellulose (HPMC), hydroxypropylmethylcellulose phthalate, a polymethylacrylic acid copolymer, or hydroxypropylmethylcellulose acetate succinate (HPMCAS).

### VII. PROCESS

The thienotriazolodiazepine compounds disclosed herein can exist as free base or as acid addition salt can be obtained according to the procedures described in US Patent Application Publication No. 2010/0286127, incorporated by reference in its entirety herein, or in the present application. Individual enantiomers and diastereomers of the thienotriazolodiazepine compounds of the present invention can be prepared synthetically from commercially available starting materials that contain asymmetric or stereogenic centers, or by preparation of racemic mixtures followed by resolution methods well known to those of ordinary skill in the art.

These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase gas chromatography or crystallizing the compound in a chiral solvent.

If desired, a particular enantiomer of the thienotriazolodiazepine compounds disclosed herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers, thus formed by fractional crystallization or chromatographic means well known in the art, and subsequent recovery of the pure enantiomers. Various methods well known in the art may be used to prepare the thienotriazolodiazepine compounds of Formula (1) with an enantiomeric excess of generally more than about 80%. Advantageously, preferred enantiomeric excess is of more than 80%, preferably of more than 90%, more preferably of more than 95%, and most preferably of 99% and more.

The solid dispersions of the present invention can be prepared by a number of methods, including by melting and solvent evaporation. The solid dispersions of the present invention can also be prepared according to the procedures described in: Chiou W L, Riegelman S: “Pharmaceutical applications of solid dispersion systems”, J. Pharm. Sci. 1971; 60:1281-1302; Serajuddin ATM: “Solid dispersion of poorly water-soluble drugs: early promises, subsequent problems, and recent breakthroughs”, J. Pharm. Sci. 1999; 88:1058-1066; Leuner C, Dressman J: “Improving drug solubility for oral delivery using solid dispersions”, Eur. J. Pharm. Biopharm. 2000; 50:47-60; and Vasconcelos T, Sarmento B, Costa P: “Solid dispersions as strategy to improve oral bioavailability of poor water soluble drugs”, Drug Discovery Today 2007; 12:1068-1075, all of which are incorporated herein by reference in their entireties.

In one embodiment, solid dispersions of the present invention are prepared by a melting process. In one embodiment, the melting process comprises melting one or more of the various embodiments of the thienotriazolodiazepine of Formula (1) within a carrier. In one embodiment, the melting process includes cooling a melted compound of the present invention and a carrier. In one embodiment, the melting process comprises pulverization of the melted compound and the carrier. In one embodiment, a melted compound of the present invention and a carrier are pulverized following the cooling step.

In some embodiments in which the thienotriazolodiazepine of Formula (1) or a pharmaceutically acceptable salt, a solvate, including a hydrate, a racemate, an enantiomer, an isomer, or an isotopically-labeled form thereof and the carrier are incompatible, a surfactant may be added during the melting step to prevent formation of two liquid phases or a suspension in the heated mixture. In some embodiments, one or more of the various embodiments of the thienotriazolodiazepine of Formula (1) is suspended in a previously melted carrier, instead of using both drug and carrier in the melted state, thereby reducing the process temperature. In one embodiment, melted drug and carrier mixture is cooled an ice bath agitation. In one embodiment, melted drug and carrier mixture is cooled and solidified by spray cooling (alternatively spray congealing).

In one embodiment, melted drug and carrier mixture is cooled and solidified by forming the melt into particles by spraying the melt into a cooling chamber through which ambient or cooled, low temperature air is passing. In one embodiment, melted drug and carrier mixture is cooled and solidified by atomization and re-solidification of the molten dispersion in a suitable fluid bed processor. In one embodiment, melted drug and carrier mixture is cooled and solidified by melt-granulation in a heatable high-shear mixer.

In some embodiments, hot-stage extrusion or melt agglomeration may be used to avoid melting limitations of the drug. Hot-stage extrusion consists of the extrusion, at high rotational speed, of the drug and carrier, previously mixed, at melting temperature for a short period of time; the resulting product is collected after cooling at room temperature and milled.

In one embodiment, one or more of the various embodiments of the thienotriazolodiazepine of Formula (1) is processed at a reduced processing temperature to avoid degradation of any thermally labile compound. In one embodiment, the reduced processing temperature is achieved by associating a hot-stage extrusion with a temporary plasticizer such as carbon dioxide. In one embodiment, melt agglomeration is used in the preparation of solid dispersions in accordance with the present invention in conventional high shear mixers or in a rotary processors. In one embodiment, the solid dispersion in accordance with the present invention is prepared by adding a molten carrier containing a thienotriazolodiazepine compound in accordance with the present invention to a heated excipient. In one embodiment, the solid dispersion in accordance with the present invention is prepared by adding by adding a molten carrier to a heated mixture of the thienotriazolodiazepine in accordance with the present invention and one or more excipients. In one embodiment, the solid dispersion in accordance with the present invention is prepared by heating a mixture of a thienotriazolodiazepine compound in accordance with the present invention, a carrier and one or more excipients to a temperature within or above the melting range of the carrier.

In some embodiments, a one or more of the various embodiments for the formulation of the thienotriazolodiazepine, according to Formula (1), is prepared by a solvent evaporation method. In one embodiment, the solvent evaporation method comprises solubilization of a thienotriazolodiazepine compound, according to Formula (1), carrier in a volatile solvent that is subsequently evaporated. In one embodiment, the volatile solvent may one or more excipients. In one embodiment, the one or more excipients include, but are not limited to anti-sticking agents, inert fillers, surfactants wetting agents, pH modifiers and additives. In one embodiment, the excipients may dissolved or in suspended or swollen state in the volatile solvent.

In one embodiment, preparation of solid dispersions in accordance with the present invention includes drying one or more excipients suspended in a volatile solvent. In one embodiment, the drying includes vacuum drying, slow evaporation of the volatile solvent at low temperature, use of a rotary evaporator, spray-drying, spray granulation, freeze-drying, or use of supercritical fluids.

In one embodiment, spray drying preparation of a formulation for the thienotriazolodiazepine composition, according to Formula (1), is used which involves atomization of a suspension or a solution of the composition into small droplets, followed by rapid removal solvent from the formulation. In one embodiment, preparation of a formulation in accordance with the present invention involves spray granulation in which a solution or a suspension of the composition in a solvent is sprayed onto a suitable chemically and/or physically inert filler, such as lactose or mannitol. In one embodiment, spray granulation of the solution or the suspension of the composition is achieved via two-way or three-way nozzles.

In some embodiments, preparation of solid dispersions in accordance with the present invention includes use of supercritical fluids. The term “supercritical fluids” refers to substances existing as a single fluid phase above their critical temperature and critical pressure. In one embodiment, preparation of a formulation, in accordance with the present invention, includes use a supercritical carbon dioxide fluid. In one embodiment, preparation of a formulation, in accordance with the present invention, using the supercritical fluid technique comprises dissolving a thienotriazolodiazepine compound, according to Formula (1), and carrier in a common solvent that is introduced into a particle formation vessel through a nozzle, simultaneously with carbon dioxide; and spraying the solution to allow the solvent be rapidly extracted by the supercritical fluid, thereby resulting in the precipitation of solid dispersion particles on the walls of the vessel.

In some embodiments, preparation of solid dispersions in accordance with the present invention includes use of a co-precipitation method. In one embodiment, a non-solvent is added dropwise to a thienotriazolodiazepine composition, according to Formula (1), and a carrier solution, under constant stirring. In one embodiment, the thienotriazolodiazepine composition, according to Formula (1), and the carrier are co-precipitated to form microparticles during the addition of the non-solvent. In one embodiment, the resulting microparticles are filtered and dried to provide the desired solid dispersion.

The invention is illustrated in the following non-limiting examples.

### VIII. EXAMPLES

### Example 1: In Vitro Screening of Solid Dispersions of Compound (1-1)

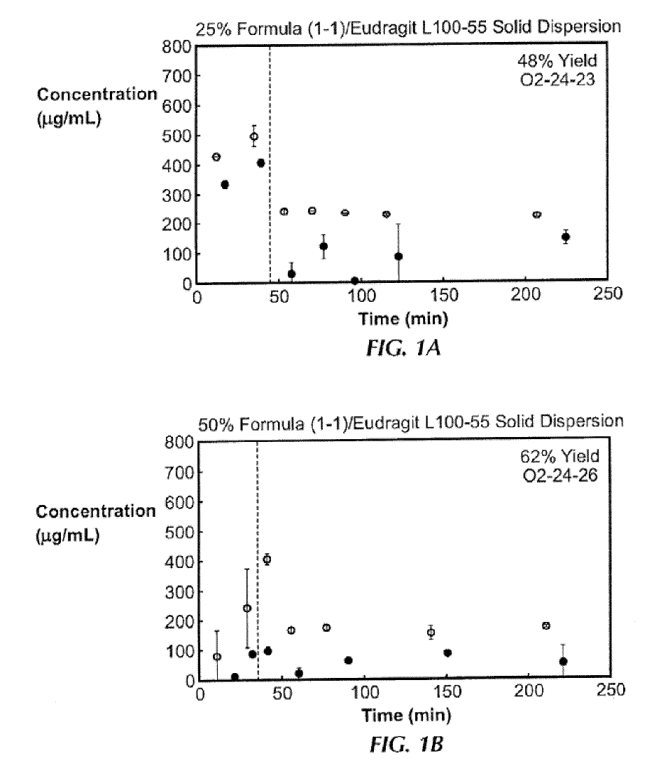

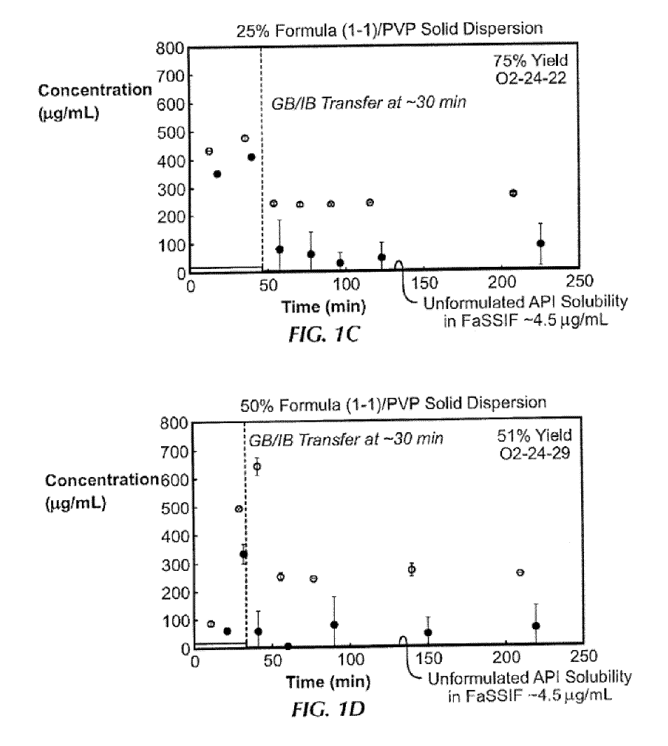

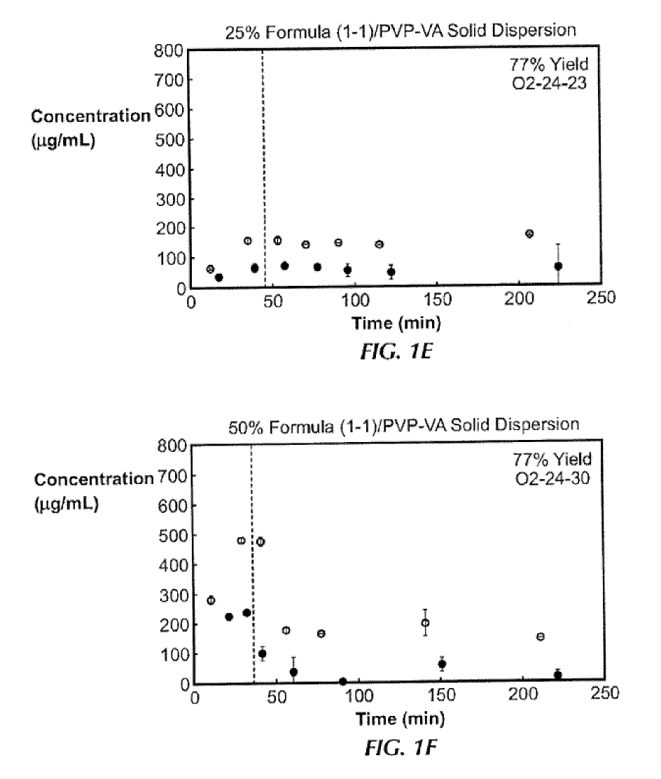

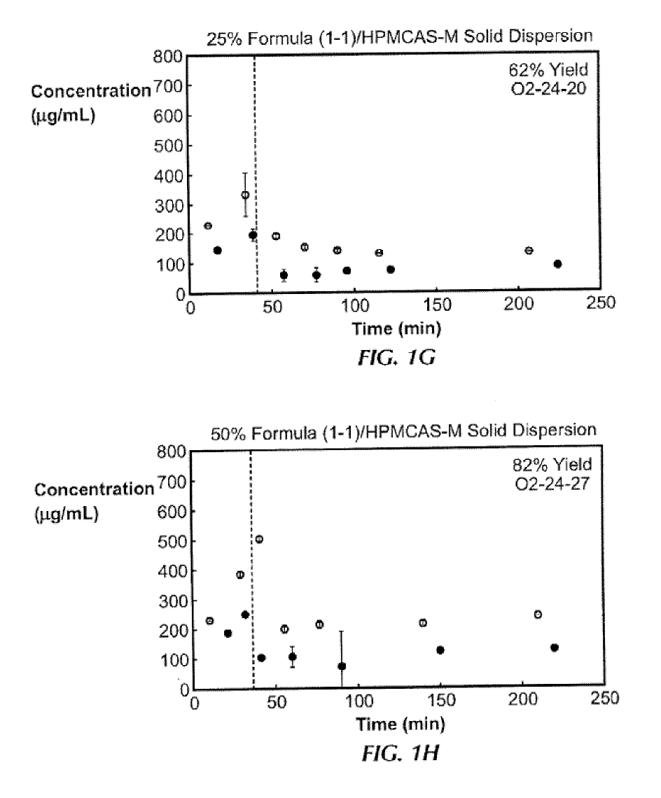

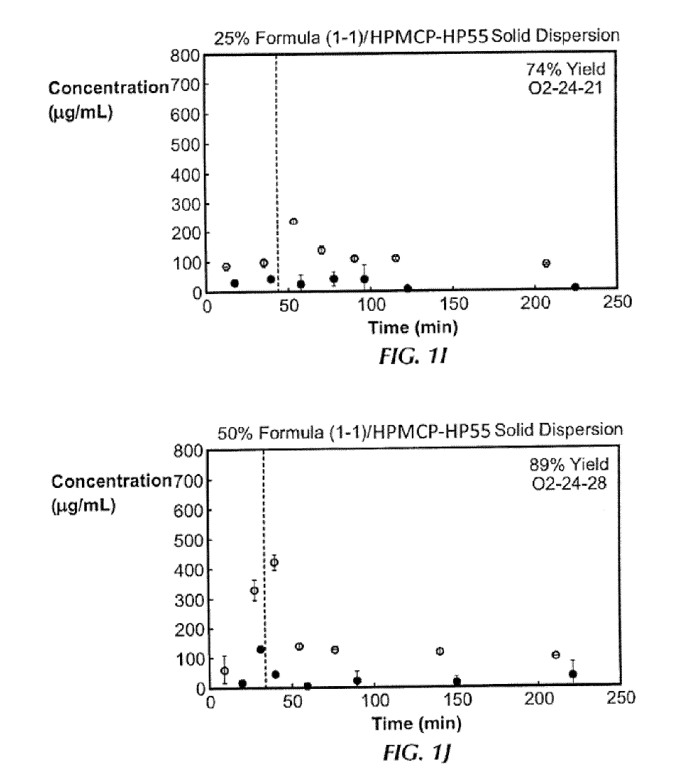

Ten solid dispersions were prepared using compound (1-1) and one of five polymers, including hypromellose acetate succinate (HPMCAS-M), hypromellose phthalate (HPMCP-HP55), polyvinylpyrrolidone (PVP), PVP-vinyl acetate (PVP-VA), and Eudragit L100-55, at both 25% and 50% of compound (1-1) loading, for each polymer. Solid dispersions were prepared by a solvent evaporation method, using spray-drying followed by secondary drying in a low-temperature convection oven. The performance of each solid dispersion was assessed via a non-sink dissolution performance test which measured both the total amount of drug and the amount of free drug present in solution over time. Non-sink dissolution was chosen because it best represents the in vivo situation for low soluble compounds. This test included a “gastric transfer” of dispersion from gastric pH (0.1N NaCl, pH 1.0) to intestinal pH (FaFSSIF, pH 6.5) approximately 30 to 40 minutes after the introduction of dispersion to the test medium, simulating in vivo conditions. [FaFSSIF is Fasted State Simulated Intestinal Fluid, comprised of 3 mM sodium taurocholate, 0.75 mM lecithin, 0.174 g NaOH pellets, 1.977 g NaH₂PO₄.H₂O, 3.093 g NaCl, and purified water qs 500 mL.] The amount of dissolved drug was quantified using a high-performance liquid chromatography (HPLC) method and an Agilent 1100 series HPLC. The dissolution profiles of the formulations (FIGS. 1A-1J) showed large increases in drug solubility in all dispersion candidates relative to the unformulated compound in the same media. Of the solid dispersions, the 25% compound (1-1) in PVP, 25% compound (1-1) in HPMCAS-M, and 50% compound (1-1) in HPMCAS-M dispersions were the most promising candidates for enhanced oral absorption as compared to the unformulated compound, based on finding higher levels of free drug released at intestinal pH.

### Example 2: In Vivo Screening of Solid Dispersions of Compound (1-1)

The three most promising solid dispersions of compound (1-1), namely the 25% compound (1-1) in PVP, 25% compound (1-1) in HPMCAS-MG, and 50% compound (1-1) in HPMCAS-M dispersions, were prepared at larger scale for in vivo studies. Each formulation was assessed in the in vitro dissolution test described in Example 1. To ensure that these dispersions were both amorphous and homogeneous, each dispersion was assessed by powder x-ray diffraction (PXRD) and modulated differential scanning calorimetry (mDSC). Additionally, to understand the effect of water on the glass transition temperature (Tg) for each dispersion, mDSC was performed on samples first equilibrated at a set relative humidity (i.e., 25%, 50%, and 75% RH) for at least 18 hours. [Water can act as a plasticizer for solid dispersions and the hygroscopicity of the system due to the active compound or polymer can affect the amount of water uptake by these systems.]

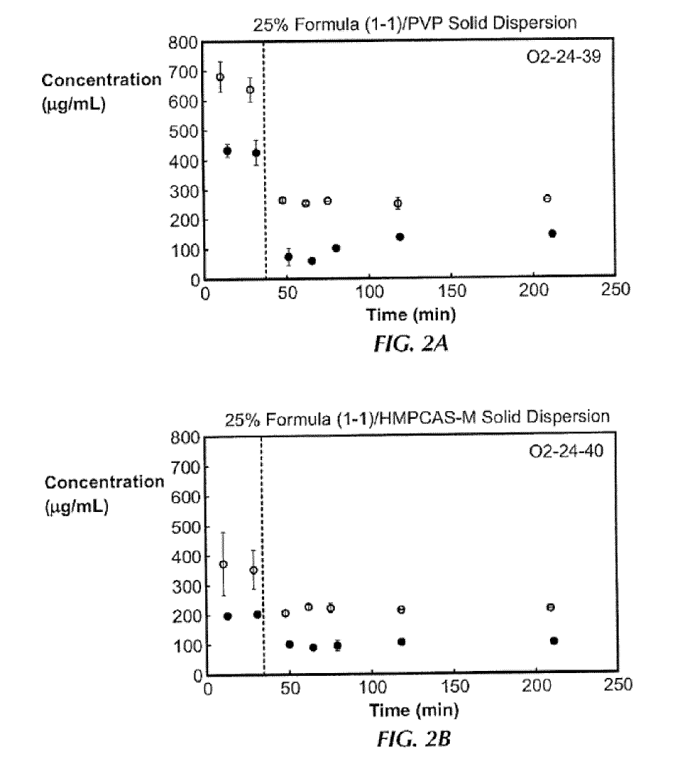

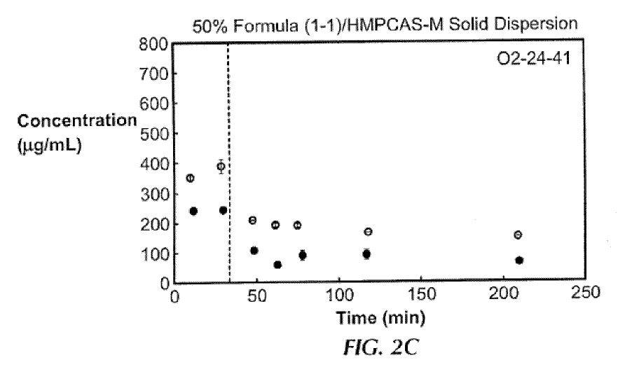

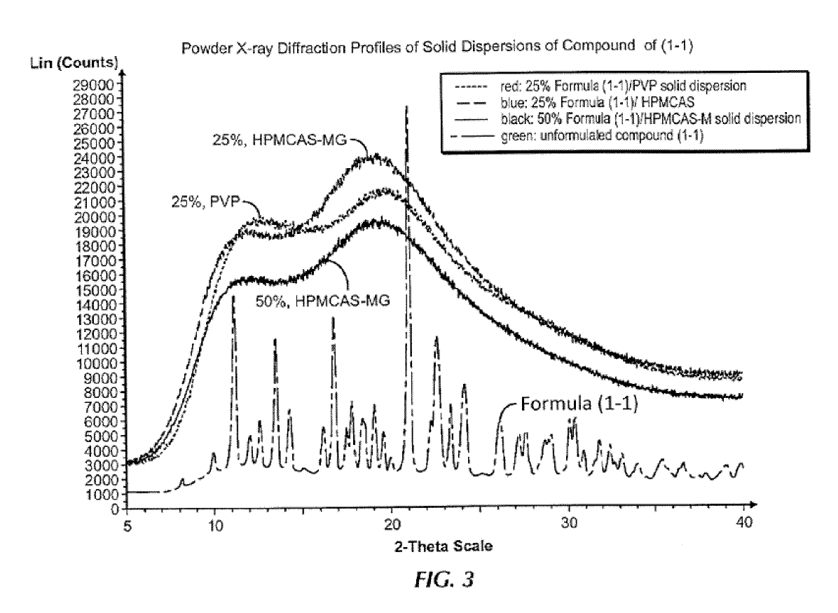

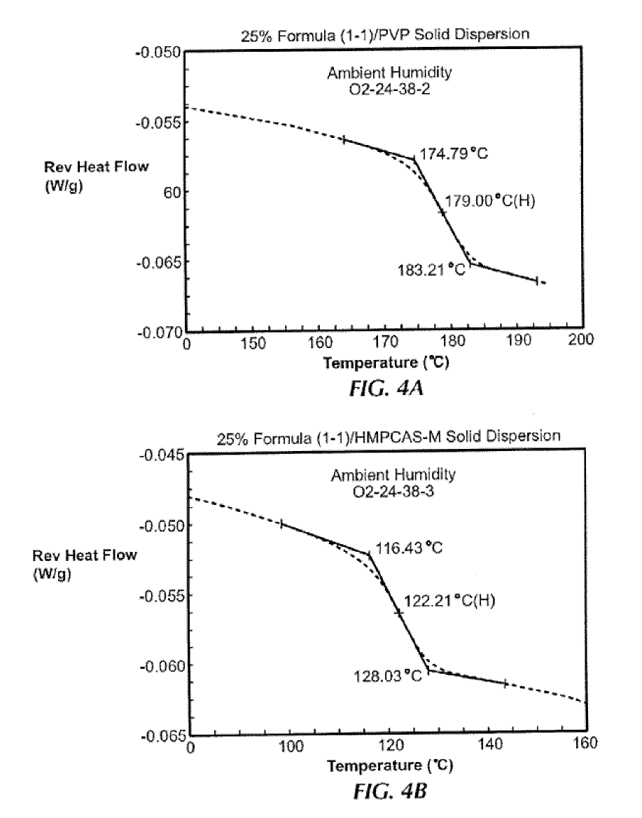

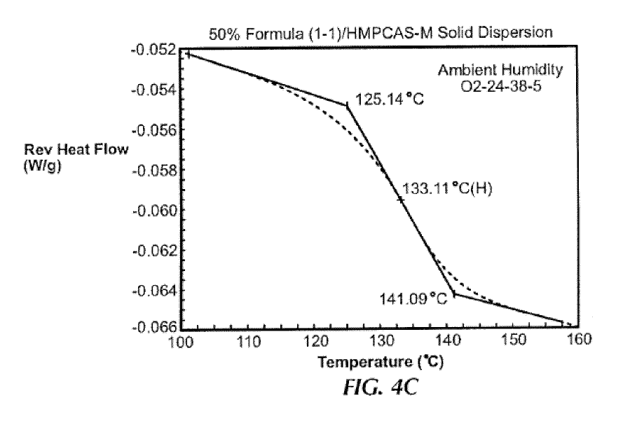

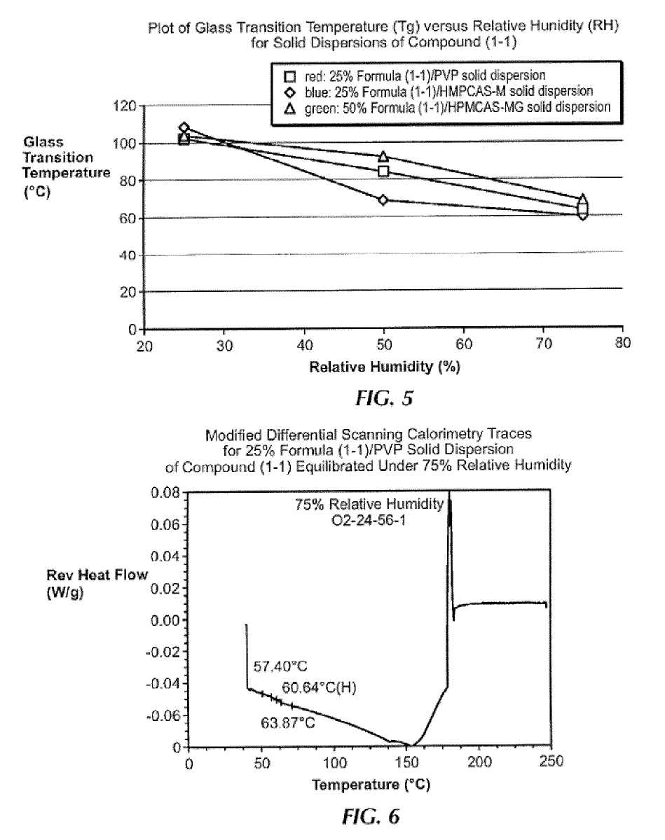

The non-sink dissolution results (FIGS. 2A-2C) were comparable to those found for the dispersions in Example 1. PXRD results (FIG. 3) showed no evidence of crystalline compound in any of the dispersions and mDSC results (FIGS. 4A-4C) showed a single glass transition temperature (Tg) for each dispersion, indicating that each dispersion was homogeneous. The x-ray diffractomer was a Bruker D-2 Phaser. An inverse relationship between Tg and relative humidity was observed for each (FIG. 5). Notably, for the 25% compound (1-1) in PVP solid dispersion equilibrated at 75% RH, there appeared to be two Tgs, indicating that phase separation was occurring, and this dispersion also showed a melt event at 75% RH, suggesting that crystallization occurred during the RH equilibration (FIG. 6). This finding suggests that the 25% compound (1-1) in PVP dispersion may be less stable than the HPMCAS-M dispersions.

To assess the bioavailability of the three dispersions, groups of male beagle dogs (three per group) were given a 3 mg/kg dose of an aqueous suspension of solid dispersion of compound (1-1) administered by oral gavage or a 1 mg/kg dose of compound (1-1) dissolved in water:ethanol:polyethylene glycol (PEG) 400 (60:20:20) and administered as an intravenous bolus into the cephalic vein. Blood samples were collected from the jugular vein of each animal at 0 (pre-dose), 5, 15, and 30 minutes and 1, 2, 4, 8, 12, and 24 hours following intravenous administration and at 0 (pre-dose), 15 and 30 minutes and 1, 2, 4, 8, 12, and 24 hours following oral gavage administration. The amount of compound (1-1) present in each sample was detected using a qualified LC-MS/MS method with a lower limit of quantification of 0.5 ng/mL. The area under the plasma concentration-time curve (AUC) was determined by use of the linear trapezoidal rule up to the last measurable concentration without extrapolation of the terminal elimination phase to infinity. The elimination half-life (t_{1/2}) was calculated by least-squares regression analysis of the terminal linear part of the log concentration-time curve. The maximum plasma concentration (Cₘₐₓ) and the time to Cₘₐₓ (tₘₐₓ) were derived directly from the plasma concentration data. The oral bioavailability (F) was calculated by dividing the dose normalized AUC after oral administration by the dose normalized AUC after intravenous administration and reported as percentages (%). Results, summarized in Table 1 below, gave mean oral bioavailabilities of the 25% compound (1-1) in PVP, 25% compound (1-1) in HPMCAS-M, and 50% compound (1-1) in HPMCAS-M solid dispersions of 58%, 49%, and 74%, respectively.

TABLE 1
pharmacokinetic parameters of compound (1-1) after
oral (po) and intravenous (iv) administrations to
dogs (the values are averages from three dogs)

| | | | | AUC | | |
|---|---|---|---|---|---|---|
| Compound (1-1) | Dose & | Cₘₐₓ | tₘₐₓ | (ng · min/ | t_{1/2} | F |
| formulation | Route | (ng/L) | (hr) | mL) | (hr) | (%) |
| Solution in | 1 mg/kg | 769 | 0.083 | 53,312 | 1.5 | — |
| water:etha- | IV | | | | | |
| nol:PEG400 | | | | | | |
| (60:20:20) | | | | | | |
| Aqueous | 3 mg/kg | 487 | 1.0 | 93,271 | 1.6 | 58 |
| suspension of | PO | | | | | |
| 25% compound | | | | | | |
| (1-1)/PVP solid | | | | | | |
| dispersion | | | | | | |
| Aqueous | 3 mg/kg | 228 | 0.5 | 78,595 | 2.0 | 49 |
| suspension of | PO | | | | | |
| 25% compound | | | | | | |
| (1-1)/HPMCAS-M | | | | | | |
| solid dispersion | | | | | | |
| Aqueous | 3 mg/kg | 371 | 1.0 | 118,174 | 1.5 | 74 |
| suspension of | PO | | | | | |
| 50% compound | | | | | | |
| (1-1)/HPMCAS-M | | | | | | |
| solid dispersion | | | | | | |

AUC: area under the plasma concentration-time curve;
Cₘₐₓ: maximum plasma concentration;
F: bioavailability;
HPMCAS: hypromellose acetate sodium;
IV: intravenous;
PEG: polyethylene glycol;
PO; per os, oral;
PVP: polyvinylpyrrolidone;
tₘₐₓ: time of Cₘₐₓ;
t_{1/2}: plasma elimination half-life

### Example 3: Preparation and Clinical Use of Capsules Containing a Solid Dispersion of Compound (1-1)

A gelatin capsule of 10 mg strength was prepared for initial clinical studies in patients with hematologic malignancies. Based on results of in vitro and in vivo testing of solid dispersions of compound (1-1), as described in Examples 1 and 2, a 50% compound (1-1) in HPMCAS-M solid dispersion was selected for capsule development. Capsule development was initiated targeting a fill weight of 190 mg in a size 3 hard gelatin capsule, as this configuration would potentially allow increasing the capsule strength by filling a larger size capsule while maintaining the pharmaceutical composition. Based on experience, four capsule formulations were designed with different amounts of disintegrant and with and without wetting agent. Since all four formulations showed similar disintegration test and dissolution test results, the simplest formulation (without wetting agent and minimum disintegrant) was selected for manufacturing. Manufacturing process development and scale-up studies were performed to confirm the spray drying process and post-drying times for the solid dispersion; blending parameters; roller compaction and milling of the blend to achieve target bulk density of approximately 0.60 g/cc; and capsule filling conditions.

Crystalline compound (1-1) and the polymer hypromellose acetate succinate (HPMCAS-M) were dissolved in acetone and spray-dried to produce solid dispersion intermediate (SDI) granules containing a 50% compound (1-1) loading. The SDI was shown by PXRD analysis to be amorphous and by mDSC analysis to be homogeneous (i.e., single Tg under ambient conditions). The 50% compound (1-1) in HPMCAS-M solid dispersion (1000 g) and excipients, including microcrystalline cellulose filler-binder (4428 g), croscarmellose sodium disintegrant (636 g), colloidal silicon dioxide dispersant/lubricant 156 g), magnesium stearate dispersant/lubricant (156 g), and lactose monohydrate filler (5364 g) were blended in stages in a V-blender. The blend was them compacted and granulated to obtain a bulk density of approximately 0.6 g/mL. The blend was dispensed into size 3 hard gelatin capsules (target fill weight: 190 mg) using an automated filling machine and finished capsules were polished using a capsule polisher machine.

Pharmacokinetic assessments were performed following oral dosing of 10 mg capsules containing the 50% compound (1-1) in HPMCAS solid dispersion and results were compared with pharmacokinetic assessments performed following oral dosing of administration of 4×10 mg capsules containing the Eudragit solid dispersion of compound (1-1) to healthy volunteers.

A comparison of the two pharmaceutical compositions is provided in Tables 2A and 2B below. The Eudragit formulation previously was described in Example 5 in US Patent Application 2009/0012064 A1, published Jan. 8, 2009. That application noted that the Eudragit solid dispersion formulation was made by dissolving and/or dispersing the thienotriazolodiazepine of formula (A) and coating excipients, including ammonio methacrylate copolymer type B (Eudragit RS), methacrylic acid copolymer type C (Eudragit L100-55), talc, and magnesium aluminosilicate, in a mixture of water and ethanol. This heterogeneous mixture then was applied to microcrystalline cellulose spheres (Nonpareil 101, Freund) using a centrifugal fluidizing bed granulator to produce granules that were dispensed into size 2 hydroxypropyl methylcellulose capsules.

In both clinical studies, blood levels of compound (1-1) were determined using validated LC-MS/MS methods and pharmacokinetic analyses were performed based on plasma concentrations of compound (1-1) measured at various time points over 24 hours after capsule administration. Results, summarized in Table 3 below, showed that the HPMCAS-M solid dispersion formulation had over 3-fold higher bioavailability in humans than the Eudragit solid dispersion formulation based on AUCs (924*4/1140, adjusting for difference in doses administered). Additionally, based on the observed Tₘₐₓ, the HPMCAS formulation is more rapidly absorbed than the Eudragit formulation (Tₘₐₓ of 1 h vs 4-6 h). The marked improvement in systemic exposure with the HPMCAS-M solid dispersion formulation is unexpected.

TABLE 2A
solid dispersion capsules of compound (1-1) for clinical use
pharmaceutical composition containing 50% HPMCAS solid dispersion
of compound (1-1): 10 mg strength, size 3 hard gelatin capsule

| | | Capsule Content | |
|---|---|---|---|
| Ingredient | Function | mg | Wt % |
| Compound of formula (II) | active agent | 10.0* | 5.56 |
| Hypromellose acetate succinate | carrier for solid | 10.0 | 5.56 |
| (HPMCAS-M) | dispersion | | |
| Lactose monohydrate | filler | 85.0 | 47.22 |
| Microcrystalline cellulose | filler-binder | 70.0 | 38.89 |
| Croscarmellose sodium | disintegrant | 10.0 | 5.56 |
| Colloidal silicon dioxide | dispersant/lubricant | 2.5 | 1.39 |
| Magnesium stearate | dispersant/lubricant | | |

| | | |
|---|---|---|
| Total | 190.0 | 100.0 |

TABLE 2B
pharmaceutical composition containing Eudragit L100-55solid dispersion
of compound (1-1): 10 mg strength, size 2 hard gelatin capsule

| | | Capsule Content | |
|---|---|---|---|
| Ingredient | Function | mg | Wt % |
| Compound (1-1) | active agent | 10.0* | 3.8 |
| Core: | | | |
| Microcrystalline cellulose | vehicle | 100.0 | 38.5 |
| spheres (Nonpareil 101, | | | |
| Freund, Inc) | | | |
| Compound/polymer layer: | | | |
| Ammonio methacrylate copolymer, | coating agent | 10.8 | 4.2 |
| type B (NF. PhEur) | | | |
| (Eudragit RS, Evonik) | | | |
| Methacrylic acid copolymer, | coating agent | 25.2 | 9.7 |
| type C (NF)/Methacrylic | | | |
| acid-ethyl acrylate | | | |
| copolymer (1:1) | | | |
| type A (PhEur) | | | |
| (Eudragit L100-55, Evonik) | | | |
| Talc | coating agent | 88.2 | 33.9 |
| Magnesium | coating agent | 20.0 | 7.7 |
| aluminometasilicate | | | |
| (Neuslin, Fuji Chemical) | | | |
| Triethyl citrate | plasticizer | 5.0 | 1.9 |
| Silicon dioxide | fluidizing agent | 0.8 | 0.3 |
| | | 260.0 | 100.0 |

*as anhydrate

TABLE 3
pharmacokinetic parameters following oral administration
of solid dispersions of compound (1-1) to humans

| | | Dose | | | |
|---|---|---|---|---|---|
| Compound (1-1) | # | and | Cₘₐₓ | Tₘₐₓ | AUC_{0-24 h} |
| formulation | Patients | Route | (ng/mL) | (hr) | (ng · h/mL) |
| Eudragit solid | 7 | 40 mg | 83 | 4 to 6 | 1140 |
| dispersion | | PO | | | |
| formulation | | | | | |
| 50% HMPCAS-M | 7 | 10 mg | 286 | 1 | 925 |
| solid dispersion | | PO | | | |
| formulation | | | | | |

AUC_{0-24 h}: area under the OTX015 plasma concentration vs. time curve over 24 hours
Cₘₐₓ: maximum concentration in plasma
hr: hour
HPMCAS: hypromellose acetate succinate
mL: milliliter
ng: nanogram
PO: per os, oral
Tₘₐₓ: time of Cₘₐₓ

### Example 4. Oral Exposure in the Rat

The oral bioavailability of three formulations of solid dispersions of compound (1-1) was determined in rats. The three dispersions chosen were the 25% dispersion of compound (1-1) in PVP, the 25% dispersion of compound (1-1) in HPMCAS-MG, and the 50% dispersion of compound (1-1) in HPMCAS-MG. The animals used in the study were Specific Pathogen Free (SPF) Hsd:Sprague Dawley rats obtained from the Central Animal Laboratory at the University of Turku, Finland. The rats were originally purchased from Harlan, The Netherlands. The rats were female and were ten weeks of age, and 12 rats were used in the study. The animals were housed in polycarbonate Makrolon II cages (three animals per cage), the animal room temperature was 21+/−3° C., the animal room relative humidity was 55+/−15%, and the animal room lighting was artificial and was cycled for 12 hour light and dark periods (with the dark period between 18:00 and 06:00 hours). Aspen chips (Tapvei Oy, Estonia) were used for bedding, and bedding was changed at least once per week. Food and water was provided prior to dosing the animals but was removed during the first two hours after dosing.

The oral dosing solutions containing the 25% dispersion of compound (1-1) in PVP, the 25% dispersion of compound (1-1) in HPMCAS-MG, and the 50% dispersion of compound (1-1) in HPMCAS-MG were prepared by adding a pre-calculated amount of sterile water for injection to containers holding the dispersion using appropriate quantities to obtain a concentration of 0.75 mg/mL of compound (1-1). The oral dosing solutions were subjected to vortex mixing for 20 seconds prior to each dose. The dosing solution for intravenous administration contained 0.25 mg/mL of compound (1-1) and was prepared by dissolving 5 mg of compound (1-1) in a mixture containing 4 mL of polyethylene glycol with an average molecular weight of 400 Da (PEG400), 4 mL of ethanol (96% purity), and 12 mL of sterile water for injection. The dosing solution containing the 25% dispersion of compound (1-1) in PVP was used within 30 minutes after the addition of water. The dosing solutions containing the 25% dispersion of compound (1-1) in HPMCAS-MG and the 50% dispersion of compound (1-1) in HPMCAS-MG were used within 60 minutes of after the addition of water. A dosing volume of 4 mL/kg was used to give dose levels of compound (1-1) of 1 mg/kg for intravenous administration and 3 mg/kg for oral administration. The dosing scheme is given in Table 4.

TABLE 4
Dosing scheme for rat oral exposure study.

| Rat | Weight | Dose (mL) | Test Item | Route |
|---|---|---|---|---|
| 1 | 236.5 | 0.95 | Compound (1-1) | intravenous |
| 2 | 221 | 0.88 | Compound (1-1) | intravenous |
| 3 | 237.5 | 0.95 | Compound (1-1) | intravenous |
| 4 | 255.5 | 1.02 | 25% dispersion of | oral |
| | | | compound (1-1) in PVP | |
| 5 | 224.2 | 0.90 | 25% dispersion of | oral |
| | | | compound (1-1) in PVP | |
| 6 | 219.2 | 0.88 | 25% dispersion of | oral |
| | | | compound (1-1) in PVP | |
| 7 | 251.6 | 1.01 | 25% dispersion of compound | oral |
| | | | (1-1) in HPMCAS-MG | |
| 8 | 240.4 | 0.96 | 25% dispersion of compound | oral |
| | | | (1-1) in HPMCAS-MG | |
| 9 | 238 | 0.95 | 25% dispersion of compound | oral |
| | | | (1-1) in HPMCAS-MG | |
| 10 | 226.6 | 0.91 | 50% dispersion of compound | oral |
| | | | (1-1) in HPMCAS-MG | |
| 11 | 228.4 | 0.91 | 50% dispersion of compound | oral |
| | | | (1-1) in HPMCAS-MG | |
| 12 | 228.5 | 0.91 | 50% dispersion of compound | oral |
| | | | (1-1) in HPMCAS-MG | |

Blood samples of approximately 50 μL were collected into Eppendorf tubes containing 5 μL of ethylenediaminetetraacetic acid (EDTA) solution at time points of 0.25, 0.5, 1, 2, 4, 8, 12, and 24 hours after dosing, with each sample collected within a window of 5 minutes from the prescribed time point. From each sample, 20 μL of plasma was obtained and stored at dry ice temperatures for analysis. Analysis of each sample for the concentration of compound (1-1) was performed using a validated liquid chromatography tandem mass spectrometry (LC-MS/MS) method with a lower limit of quantitation of 0.5 ng/mL.

Pharmacokinetic parameters were calculated with the Phoenix WinNonlin software package (version 6.2.1, Pharsight Corp., CA, USA) with standard noncompartmental methods. The elimination phase half-life (t_{1/2}) was calculated by least-squares regression analysis of the terminal linear part of the log concentration-time curve. The area under the plasma concentration-time curve (AUC) was determined by use of the linear trapezoidal rule up to the last measurable concentration and thereafter by extrapolation of the terminal elimination phase to infinity. The mean residence time (MRT), representing the average amount of time a compound remains in a compartment or system, was calculated by extrapolating the drug concentration profile to infinity. The maximum plasma concentration (Cₘₐₓ) and the time to Cₘₐₓ (tₘₐₓ) were derived directly from the plasma concentration data. The tentative oral bioavailability (F) was calculated by dividing the dose normalised AUC after oral administration by the dose normalised AUC after intravenous administration, i.e. F=(AUC(oral)/Dose(oral))/(AUC(intravenous)/Dose(intravenous))] and is reported as percentage (%).

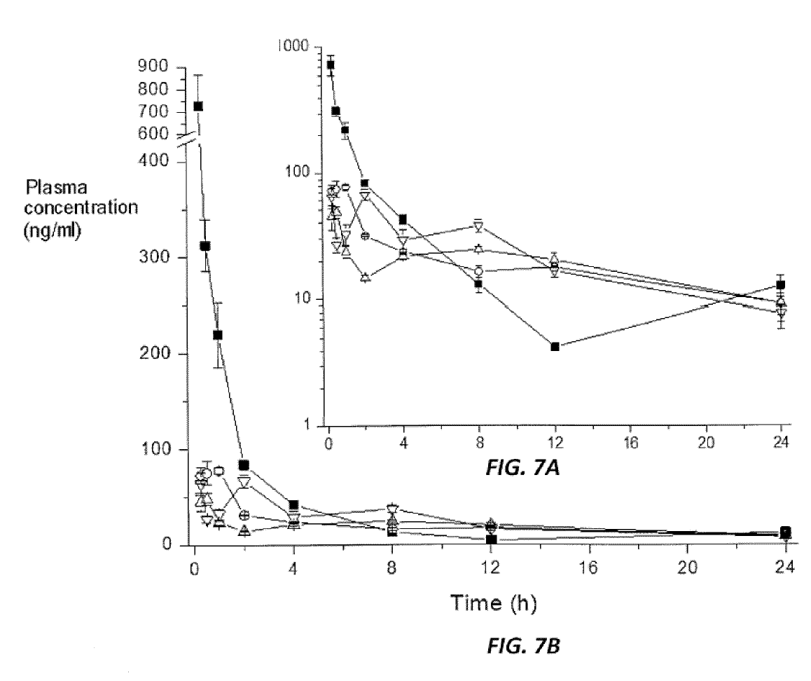

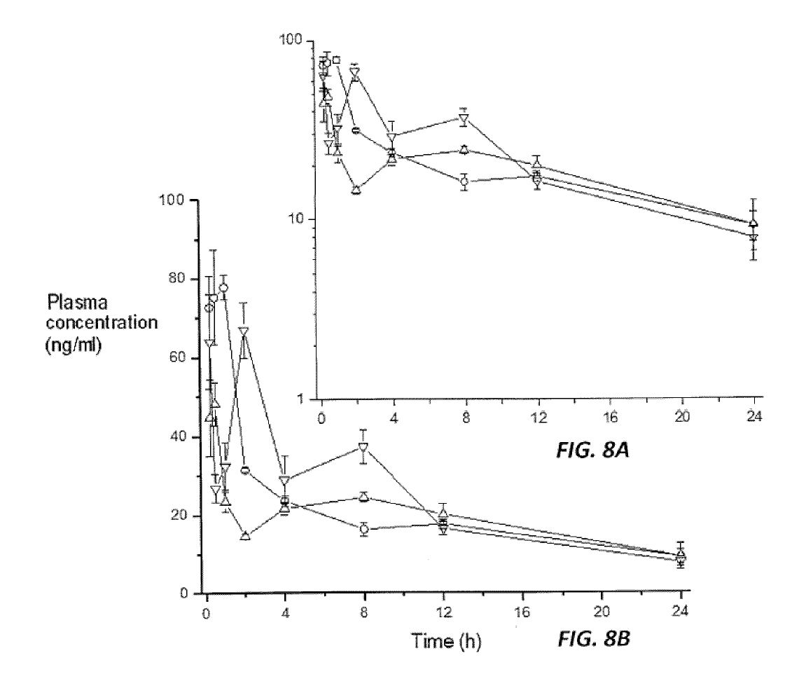

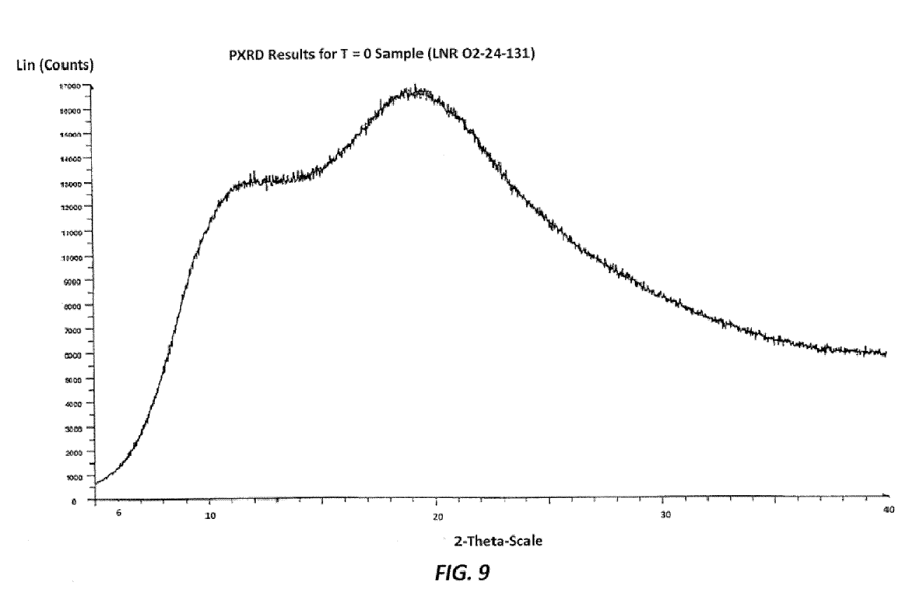

The pharmacokinetic parameters are given in Table 5, and the plasma concentration versus time plots are shown in FIGS. 7A, 7B, 8A, and 8B.

TABLE 5
Pharmacokinetic parameters of compound (1-1)
after oral and intravenous administrations. The
values are an average from three animals.

| | | 1 mg/kg | 3 mg/kg | |
|---|---|---|---|---|
| Compound | Parameter | intravenous | oral | F(%) |
| Compound (1-1) | AUC (min*ng/ml) | 74698 | | |
| water:ethanol:PEG | Cₘₐₓ (ng/ml) | 730 | | |
| 400 (60:20:20) | Tₘₐₓ (hr) | 0.25 | | |
| | t_{1/2} (hr) 8.5 | 8.5 | | |
| | CI/F (ml/min/kg) | 13.4 | | |
| | MRT (hr) | 7.4 | | |
| 25% dispersion of | AUC (min*ng/ml) | | 39920 | 18 |
| compound (1-1) in | Cₘₐₓ (ng/ml) | | 77.9 | |
| PVP | Tₘₐₓ (hr) | | 1 | |
| | t_{1/2} (hr) 8.5 | | 13.8 | |
| | CI/F (ml/min/kg) | | 75.2 | |
| | MRT (hr) | | 18.0 | |
| 25% dispersion of | AUC (min*ng/ml) | | 35306 | 16 |
| compound (1-1) in | Cₘₐₓ (ng/ml) | | 48.3 | |
| HPMCAS-MG | Tₘₐₓ (hr) | | 0.5 | |
| | t_{1/2} (hr) 8.5 | | 11.0 | |
| | CI/F (ml/min/kg) | | 85.0 | |
| | MRT (hr) | | 17.1 | |
| 50% dispersion of | AUC (min*ng/ml) | | 40238 | 18 |
| compound (1-1) in | Cₘₐₓ (ng/ml) | | 67.0 | |
| HPMCAS-MG | Tₘₐₓ (hr) | | 2 | |
| | t_{1/2} (hr) 8.5 | | 9.5 | |
| | CI/F (ml/min/kg) | | 74.6 | |
| | MRT (hr) | | 12.8 | |

### Example 5. Preparation of Spray Dried Dispersions

Spray dried dispersions of compound (1-1) were prepared using five selected polymers: HPMCAS-MG (Shin Etsu Chemical Co., Ltd.), HPMCP-HP55 (Shin Etsu Chemical Co., Ltd.), PVP (ISP, a division of Ashland, Inc.), PVP-VA (BASF Corp.), and Eudragit L100-55 (Evonik Industries AG). All spray dried solutions were prepared at 25% and 50% by weight with each polymer. All solutions were prepared in acetone, with the exception of the PVP solutions, which were prepared in ethanol. For each solution, 1.0 g of solids (polymer and compound (1-1)) were prepared in 10 g of solvent. The solutions were spray dried using a Büchi B-290, PE-024 spray dryer with a 1.5 mm nozzle and a Büchi B-295, P-002 condenser. The spray dryer nozzle pressure was set to 80 psi, the target outlet temperature was set to 40° C., the chiller temperature was set to −20° C., the pump speed was set to 100%, and the aspirator setting was 100%. After spray drying, the solid dispersions were collected and dried overnight in a low temperature convection oven to remove residual solvents.

### Example 6. Stability with Humidity and Temperature

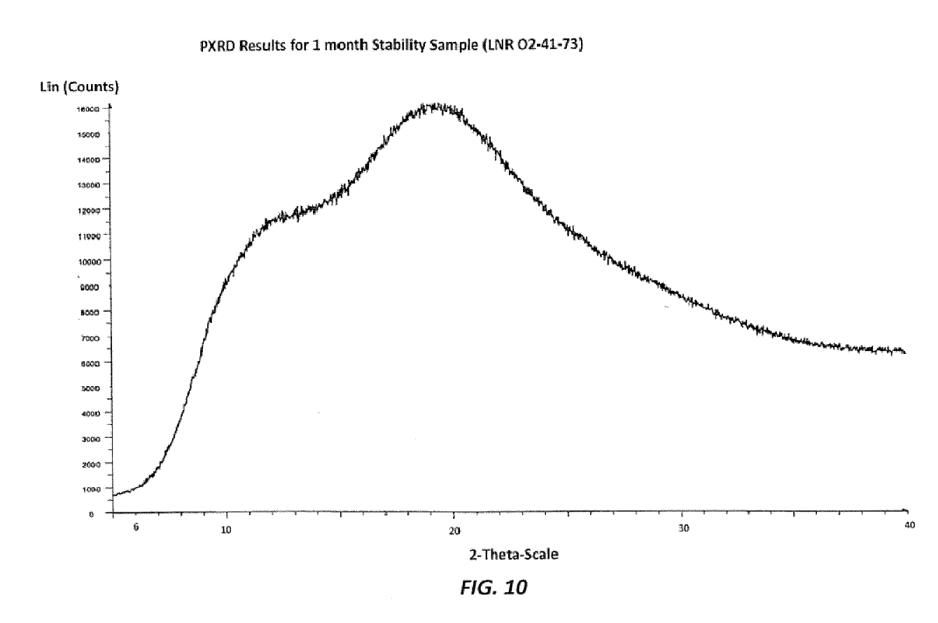

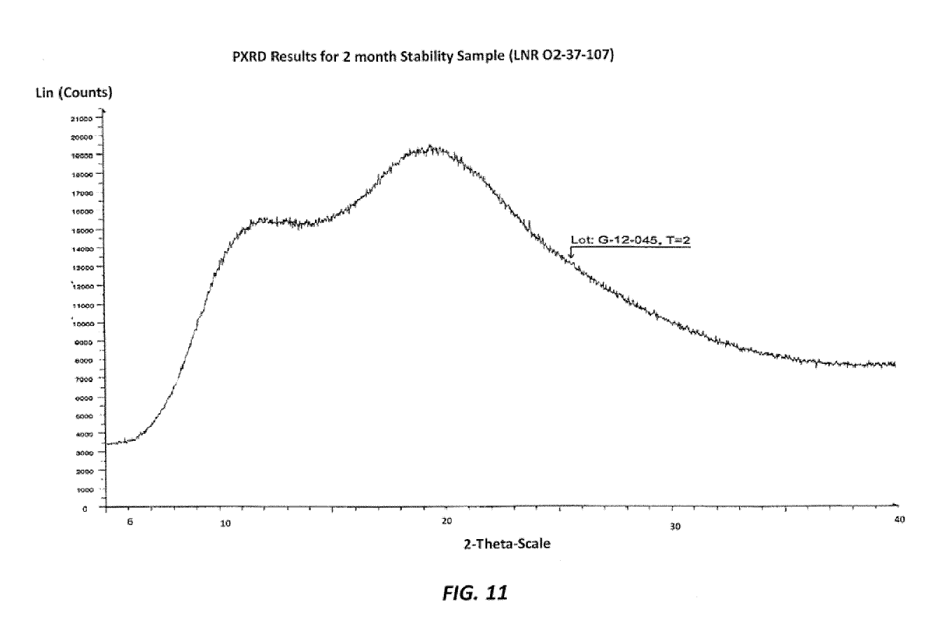

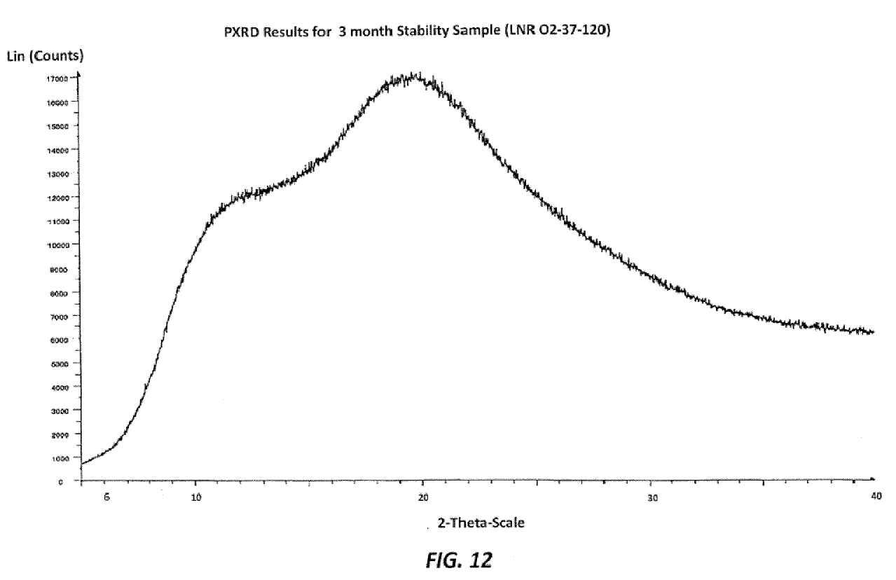

TABLE 6

| | | Accep- | | | T = 1 month | | T = 2 month | | T = 3 month | |
|---|---|---|---|---|---|---|---|---|---|---|
| | Proce- | tance | T = 0 | | (storage at | | (storage at | | (storage at | |
| Test | dure | Criteria | (Initial) | | 40° C./75% RH) | | 40° C./75% RH) | | 40° C./75% RH) | |
| Appear- | AM- | White to | Test Date/Ref: | | Test Date/Ref: | | Test Date/Ref: | | Test Date/Ref: | |
| ance | 0002 | off-white | 6 Aug. 2012/02-41-2 | | 24 Sep. 2012/02-41-59 | | 24 Oct. 2012/02-37-106 | | 17 Dec. 2012/02-37-119 | |
| | | powder | White Powder | | White Powder | | White Powder | | White Powder | |
| Potency | AM- | 45.0 · | Test Date/Ref: | | Test Date/Ref: | | Test Date/Ref: | | Test Date/Ref: | |
| (HPLC) | 0028 | 55.0 wt % | 25 Jul. 2012/02-37-21 | | 25 Sep. 2012/02-4H10 | | 24 Oct. 2012/02-37-105 | | 29 Nov. 2012/02-34-107 | |
| | | | 50.0 | | 49.4 | | 49.8 | | 49.2 | |
| Individual | AM- | Report | Test Date/Ref: | | Test Date/Ref: | | Test Date/Ref: | | Test Date/Ref: | |
| Related | 0029 | results | 25 Jul. 2012/02•34-49 | | 26 Sep. 2012/02-41-64 | | 24 Oct. 2012/02-37-105 | | 29 Nov. 2012/02-34-107 | |
| Sub- | | | RRT | % Area | RRT % | Area | RRT | % Area | RRT | % Area |

| | | | | | | |
|---|---|---|---|---|---|---|
| stances | No reportable | No reportable | 0.68 | 0.06 | 0.68 | 0.07 |
| (HPLC) | related substances | related substances | 0.77 | 0.06 | 0.77 | 0.09 |

| | | | | | | |
|---|---|---|---|---|---|---|
| Total | AM•0029 | Report | Test Date/Ref: | Test Date/Ref: | Test Date/Ref: | Test Date/Ref: |
| Related | | results | 25 Jul. 2012/02-34-49 | 26 Sep. 2012/02-41-64 | 24 Oct. 2012/02-37-105 | 29 Nov. 2012/02-34-107 |
| Sub- | | | No reportable | No reportable | 0.12% | 0.16% |
| stances | | | related substances | related substances | | |
| (HPLC) | | | | | | |
| Water | AM- | Report | Test Date/Ref: | Test Date/Ref: | Test Date/Ref: | Test Date/Ref: |
| Content | 0030 | results | 2 Aug. 2012/02-41-1 | 27 Sep. 2012/02-37-99 | 25 Oct. 2012102-37-110 | 29 Nov. 2012/02•37-116 |
| (KF) | USP | (wt %) | 1.52 | 2.53 | 2.70 | 3.43 |
| | <921> | | | | | |
| X-Ray | USP | Consistent | Test Date/Ref: | Test Date/Ref: | Test Date/Ref: | Test Date/Ref: |
| Powder | <941> | with an | 24 Jul. 2012/02•24-131 | 1 Oct. 2012/02-41-73 | 24 Oct. 2012/02-37-107 | 17 Dec. 2012/02-37-120 |
| Diffrac- | | amor- | Consistent with an | Consistent with an | Consistent with an | Consistent with an |
| tion | | phous | amorphous form | amorphous form | amorphous form | amorphous form |
| (XRPD) | | form | See FIG. 9 | See FIG. 10 | See FIG. 11 | See FIG. 12 |
| Modulated | USP | Report | Test Date/Ref: | Test Date/Ref: | Test Date/Ref: | Test Date/Ref: |
| Differ- | <891> | individual | 24 Jul. 2012/02-24-130 | 26 Sep. 2012/02-37-98 | 24 Oct. 2012/02-37-108 | 17 Dec. 2012/02-37-121 |
| ential | (n = 2 | and | Replicate 1 = | Replicate 1 = | Replicate 1 = | Replicate 1 = |
| Scanning | repli- | average | 134.30° C., | 134.65° C., | 135.35° C., | 134.36° C. |
| Calorimetry | cates) | glass | Replicate 2 = | Replicate 2 = | Replicate 2 = | Replicate 2 = |
| (mDSC) | | transition | 134.23° C., | 134.43° C., | 134.93° C., | 137.16° C. |
| | | temperatures | Replicate 3 = | Average = | Average = | Average = |
| | | (Tg, ° C.) | 135.28° C., | 134.54° C. | 135.14° C. | 135.76° C. |
| | | | Average = | | | |
| | | | 134.60° C. | | | |

Spray dried dispersions of compound (1-1) in HPMCAS-MG were assessed for stability by exposure to moisture at elevated temperature. The glass transition temperature (Tg) as a function of relative humidity was determined at 75% relative humidity, 40° C. for 1, 2 and 3 months. The spray dried dispersion was stored in an LDPE bag inside a HDPE bottle to simulate bulk product packaging. The data is summarized in Table 6. At time zero, the Tg was 134° C., at 1 month the Tg was 134° C., at 2 months the Tg was 135° C. and at 3 months the Tg was 134° C. and only a single inflection point was observed for each measurement. X-ray diffraction patterns were also obtained for each sample. FIG. 9 illustrates a powder X-ray diffraction profile of solid dispersions of compound (1-1) in HPMCAS-MG at time zero of a stability test. FIGS. 10, 11 and 12 illustrate powder X-ray diffraction profiles of solid dispersions of compound (1-1) in HPMCAS-MG after 1 month, 2 months and 3 months, respectively, after exposure at 40° C. and 75% relative humidity. The patterns did not show any diffraction lines associated with compound (1-1).

### Example 7: Compound (1-1) and Expression of c-MYC and HEXIM1

Methods:

c-MYC, BRD2/3/4 and HEXIM1 expression was assessed in six acute myeloid leukemia (AML; K562, HL-60, NB4, NOMO-1, KG1, OCI-AML3) and two acute lymphoid leukemia (ALL; JURKAT and RS4-11) cell lines after exposure to 500 nM compound (1-1). Quantitative RT-PCR and Western blotting were performed at different time points (24-72 h). A heatmap was computed with R-software.

Results:

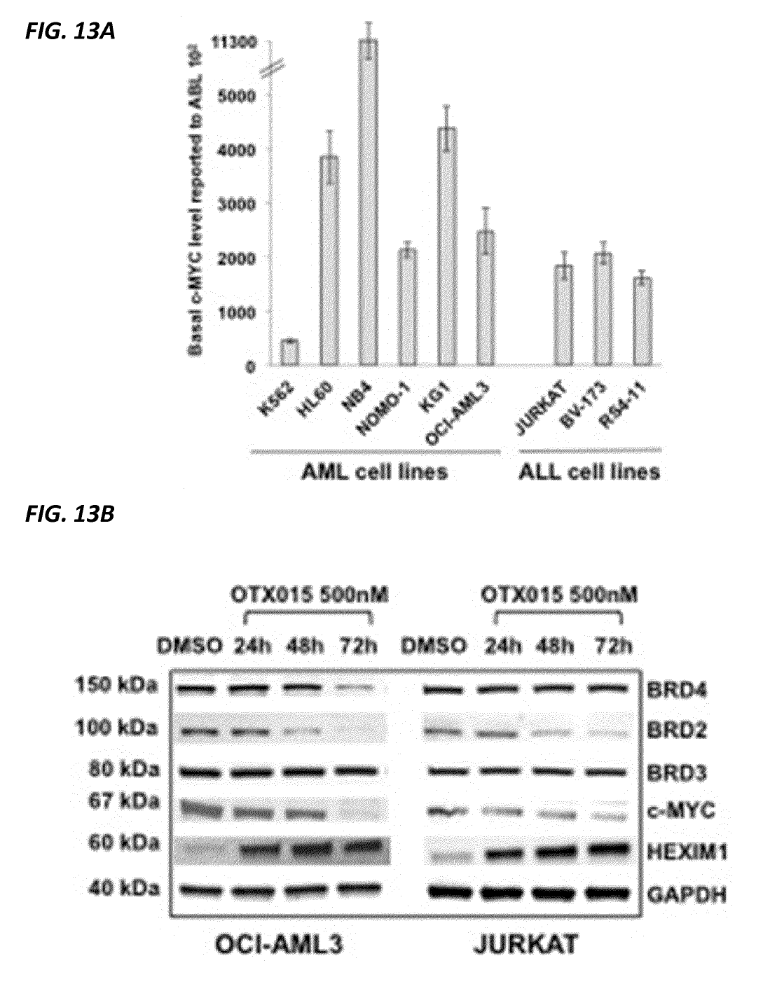

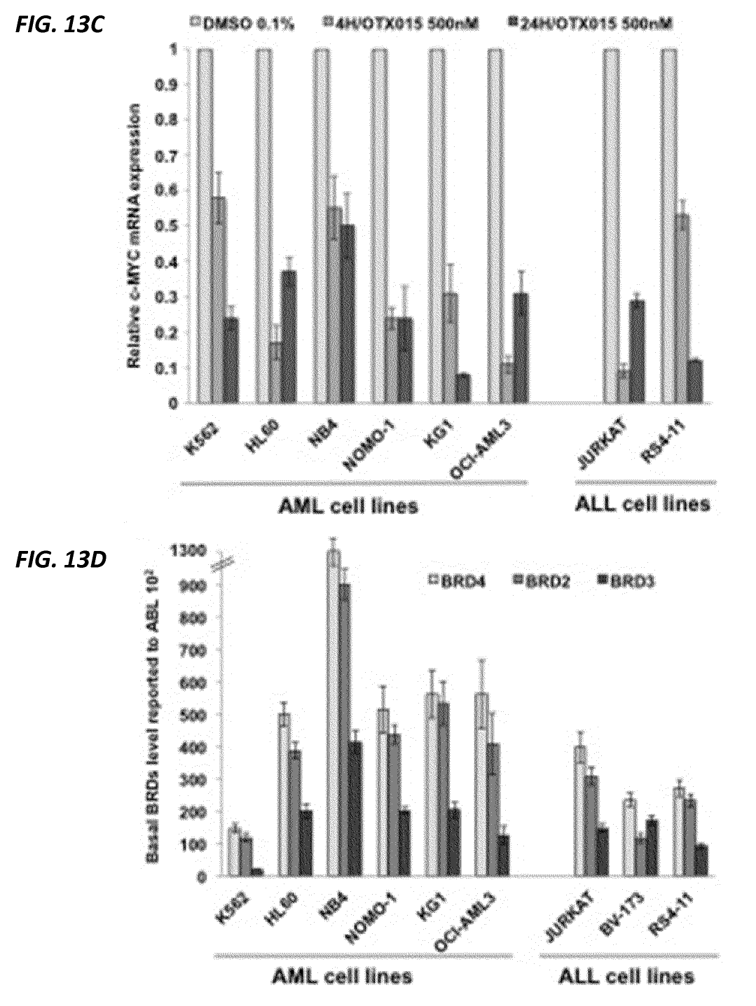

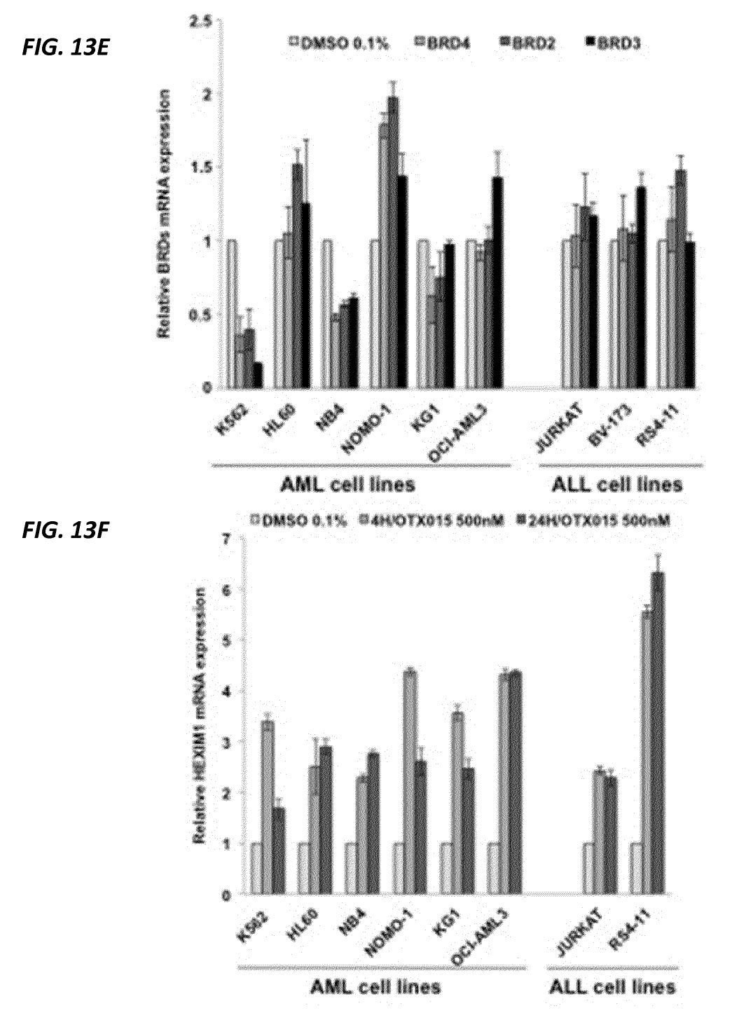

c-MYC RNA levels were ubiquitously downregulated in all AML and ALL cell lines after 24 h exposure to compound (1-1) (FIG. 13). c-MYC protein levels decreased to a variable extent at 24-72 h in all cell lines evaluated other than KG1. BRD2, BRD3 and BRD4 mRNA expression was significantly decreased in K562 cells (known to be compound (1-1)-resistant) after 48 h exposure to compound (1-1) but was increased in HL60 and NOMO-1 cells, while minimal to no increases were observed in other cell lines. Compound (1-1) induced a decrease in BRD2 protein expression in most cell lines, but not in K562 cells. In contrast, decreased BRD4 protein expression was only seen in the OCI-AML3, NB4 and K562 cell lines. BRD3 protein levels were unmodified after compound (1-1) exposure in all cell lines evaluated other than KG1. HEXIM1 mRNA expression increased after 24 h exposure to 500 nM compound (1-1) in all cell lines except compound (1-1) resistant K562 cells in which the increase was considered insignificant (less than twice). Increases in HEXIM1 protein levels were observed in OCI-AML3, JURKAT and RS4-11 cell lines at 24-72 h but not in K562 cells.

Taken together, these results show that BRD inhibition by compound (1-1) modulates the gene and protein expression of HEXIM1, in addition to c-MYC decrease and BRD variations. HEXIM1 upregulation seems to be restricted to compound (1-1) sensitive cell lines and was not significantly affected in compound (1-1)-resistant K562 cells. Further studies are needed to clarify the role of HEXIM1 in antileukemic activity of BRD inhibitors.

### Example 8: Effects of Compound (1-1) on c-MYC, BRD2/3/4 and HEXIM1 in Acute Leukemia Cell Lines

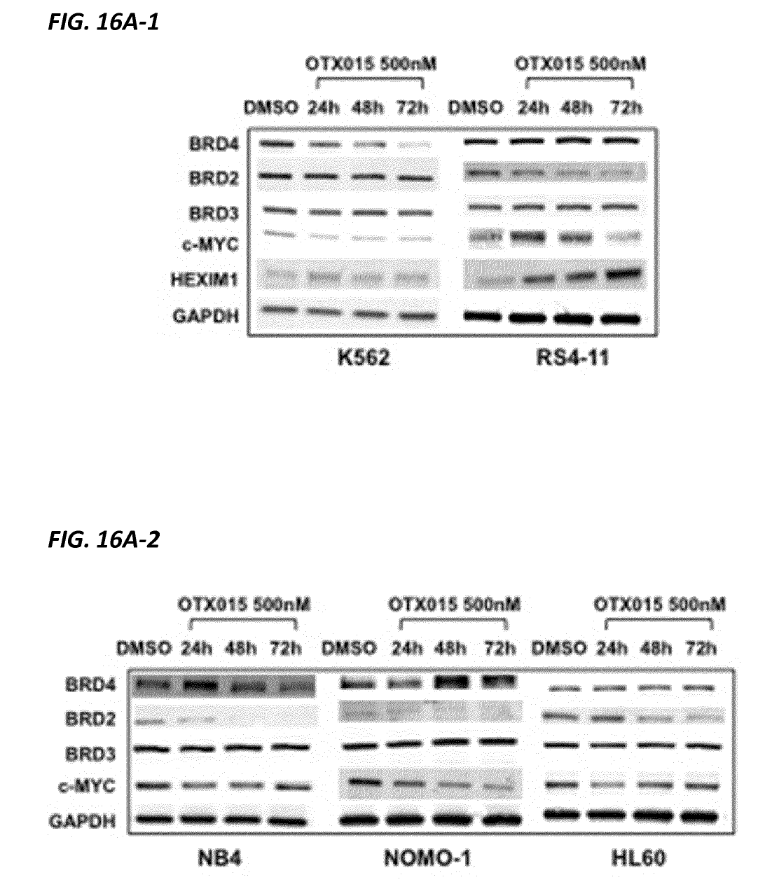

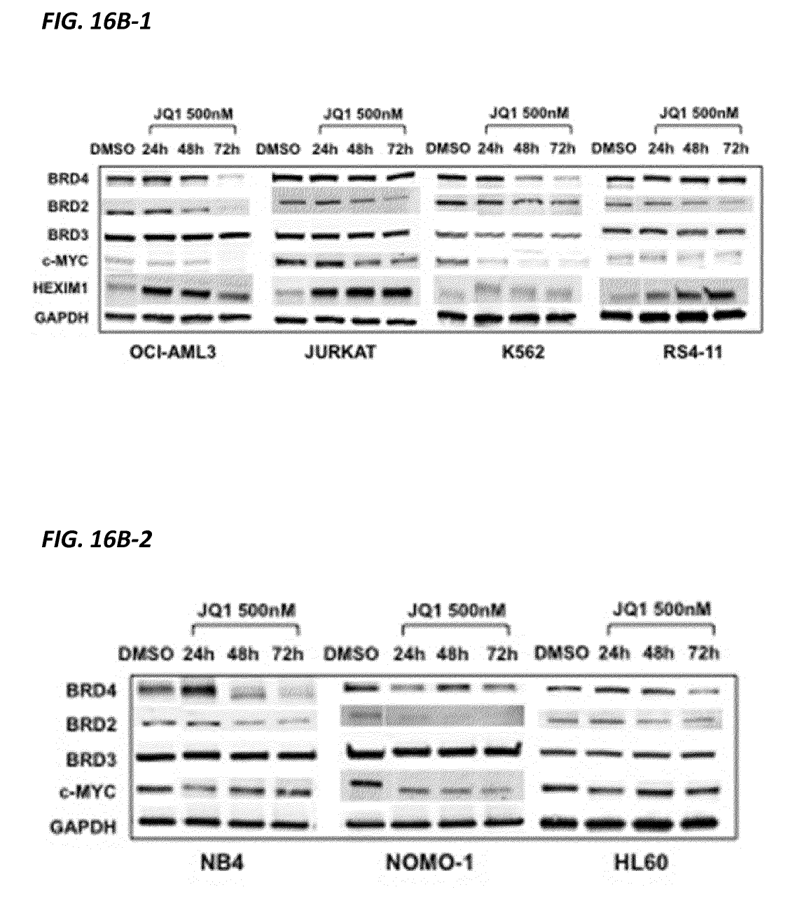

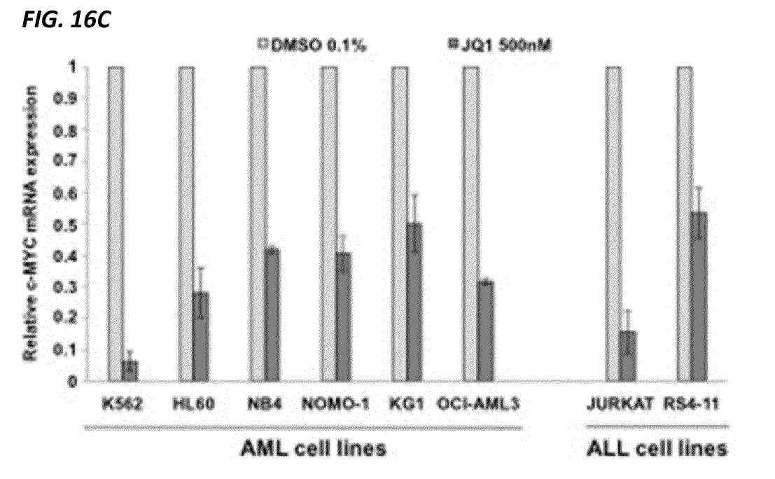

The effect of 500 nM compound (1-1) for 4, 24, 48 or 72 h on c-MYC protein and mRNA expression in a panel of AL cell lines was evaluated. Basal c-MYC gene expression varied among cell lines, with lowest levels in BCR-ABL+K562 cells and highest levels in PML-RARα rearranged NB4 (FIG. 13A). Following exposure to compound (1-1), c-MYC protein and mRNA expression was analyzed. Decreases in c-MYC protein were observed to a variable extent as early as 24 h after treatment in all cell lines tested, including AML cell lines (NPM1-mutated OCI-AML3, BCR-ABL+K562, PML-RARα-rearranged NB4, MLL-AF9 fused NOMO 1 and NRAS-driven HL60), and ALL cell lines (T-ALL JURKAT and MLL-AF4 fused B-ALL RS4-11 cells) (FIG. 13B, FIG. 16A-1, FIG. 16A-2). In line with these results, c-MYC gene expression decreased ubiquitously after 4 h and 24 h compound (1-1) exposure in these cell lines as well as in the OP2-FGFR1 rearranged KG1 AML cell line (FIG. 13C). Treatment of these cell lines with 500 nM JQ1 gave decreases in c-MYC protein similar to those seen with compound (1-1) at 24, 48 and 72 h, as well as in c-MYC gene expression at 48 h in all cell lines tested (FIG. 16B-1, FIG. 16B-2 and FIG. 16C, respectively).

The effect of compound (1-1) exposure on gene and protein expression of BRDs was also determined. Among AML cell lines, basal gene expression levels of BRDs were lowest in the BCR-ABL+K562 cell line and highest in PML-RARα rearranged NB4 (FIG. 13D). After exposure to 500 nM compound (1-1) for 48 h BRD2, BRD3 and BRD4 mRNA expression dramatically decreased in the K562 and NB4 cell lines but increased in HL60 and NOMO-1 cells (FIG. 13E). Only mild variations of BRD2, BRD3 and BRD4 mRNA expression were observed in KG1, OCI-AML3, JURKAT, BV-173 and RS4-11. Compound (1-1) induced a decrease in BRD2 protein expression in most cell lines, including OCI-AML3, JURKAT T-ALL, RS4-11, NB4, NOMO-1 and HL60 cells but not in K562 cells (FIG. 13B and FIG. 16A-1, FIG. 16A-2). In contrast, decreased protein expression of BRD4 protein after compound (1-1) treatment was only seen in the OCI-AML3, NB4 and K562 cell lines. Finally, BRD3 protein levels were unmodified after compound (1-1) exposure in all cell lines analyzed (FIG. 13B and FIG. 16-A1, FIG. 16A-2). Compared to compound (1-1), treatment with JQ1 induced a similar profile of BRD2, BRD3 and BRD4 protein modulation (FIG. 16-B1, FIG. 16B-2).

The effect of compound (1-1) on HEXIM1 expression was evaluated. HEXIM1 mRNA expression was increased after both 4 and 24 h compound (1-1) exposure at 500 nM in all cell lines tested (K562, HL-60, NB4, NOMO-1, KG1, OCI-AML3, JURKAT and RS4-11; FIG. 13F). HEXIM1 upregulation after compound (1-1) exposure was highest in OCI-AML3 and RS4-11 cell lines. Treatment with either compound (1-1) or JQ1 at 500 nM (24-72 h) yielded a similar increase in HEXIM1 protein levels after 24, 48 and 72 h in OCI-AML3, JURKAT and RS4-11 cell lines but not in K562 cells (FIG. 13B and FIG. 16A-1, FIG. 16A-2, FIG. 16B-1, and FIG. 16B-2).

### Example 9: Effect of Compound (1-1) on Cell Proliferation, Cell Cycle and Apoptosis in Leukemia Cell Lines

Cellular effects of compound (1-1) in various acute leukemia subtypes were evaluated. Cell viability after compound (1-1) exposure was evaluated with the MTT assay in nine AML and four ALL cell lines. Significant growth inhibition, defined as submicromolar IC50, was found in six of nine AML cell lines and all four ALL cell lines tested. The K562, KG1a and HL60 AML cell lines were resistant to compound (1-1).

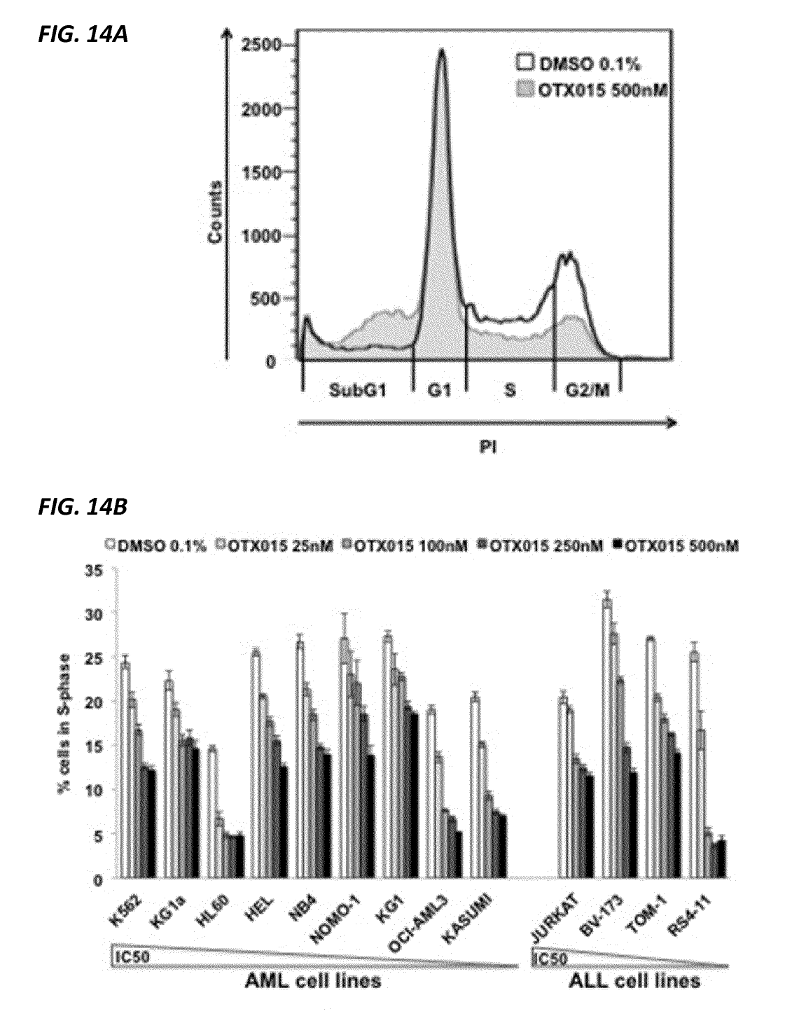

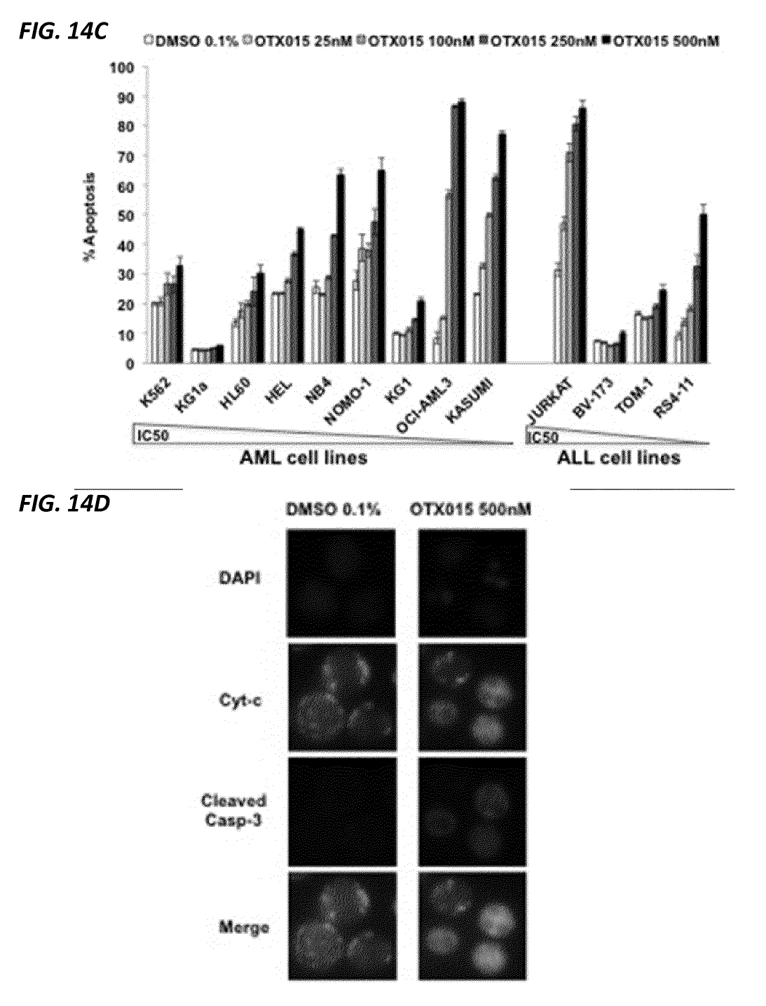

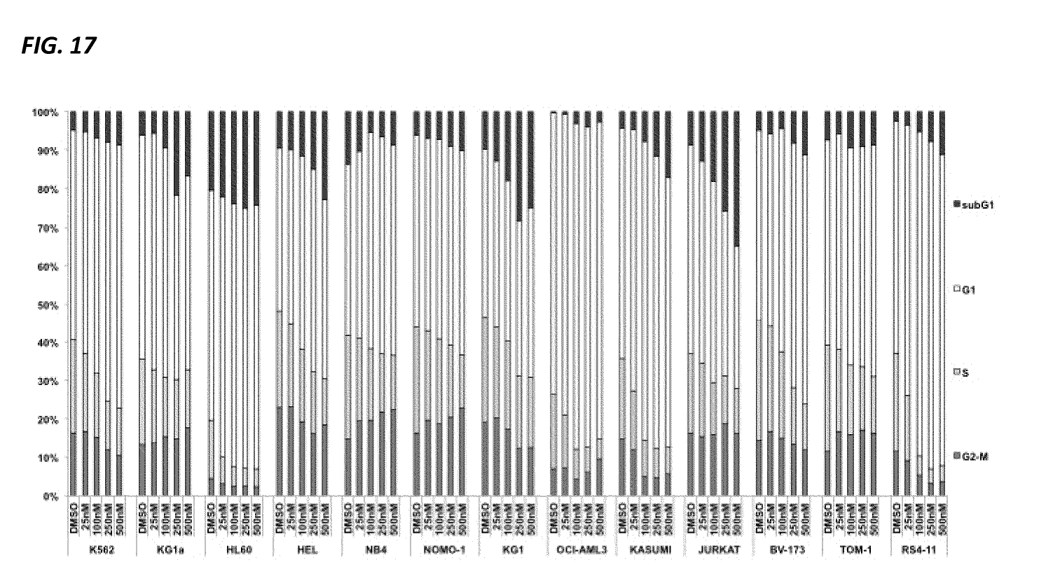

The effect of 500 nM compound (1-1) for 48 h on the cell cycle resulted in decreased transition from G1 to S-phase in all 13 cell lines and a significant increase in cells in the sub-G1 phase in KG1a, KG1, HEL, KASUMI and JURKAT cell lines (FIGS. 14A, 14B and 17).

Treatment with compound (1-1) at doses from 25 to 500 nM for 72 h induced significant apoptosis, as detected by Annexin V staining and PI uptake. At 500 nM compound (1-1), 30-90% of cells were apoptotic in five of nine AML cell lines (HEL, NB4, NOMO-1, OCI-AML3, KASUMI) and 50-90% in two of four ALL cell lines (JURKAT and RS4-11; FIG. 14C). Finally, 72 h exposure to 500 nM compound (1-1) activated caspase-3 and induced cytochrome c release, suggesting that BET inhibition leads at least in part to mitochondrial triggered apoptosis (FIG. 14D).

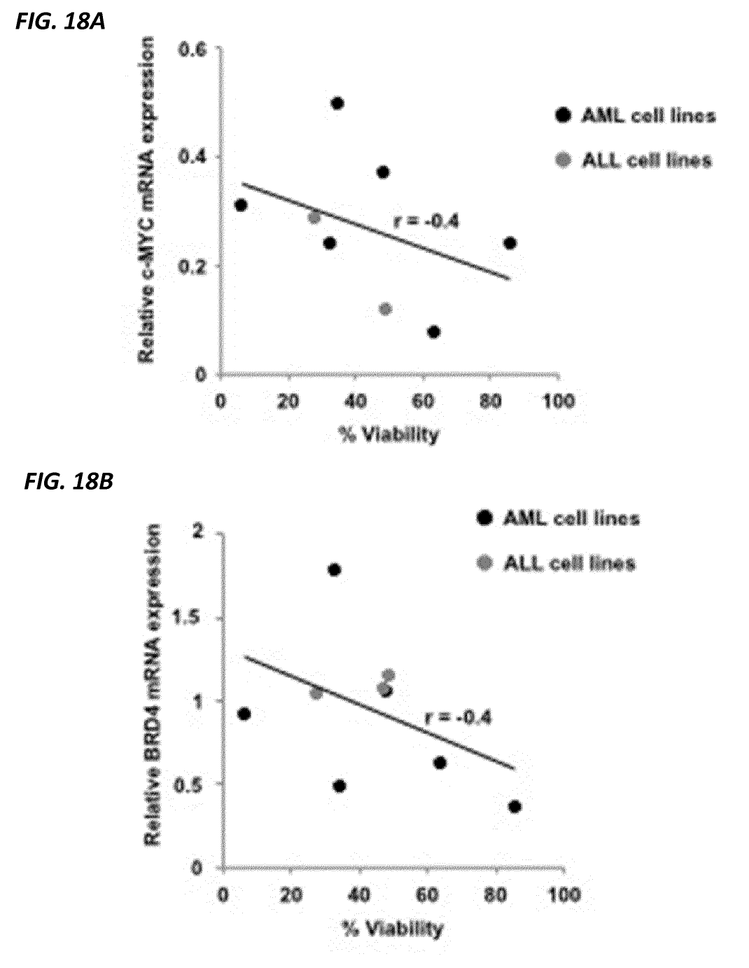

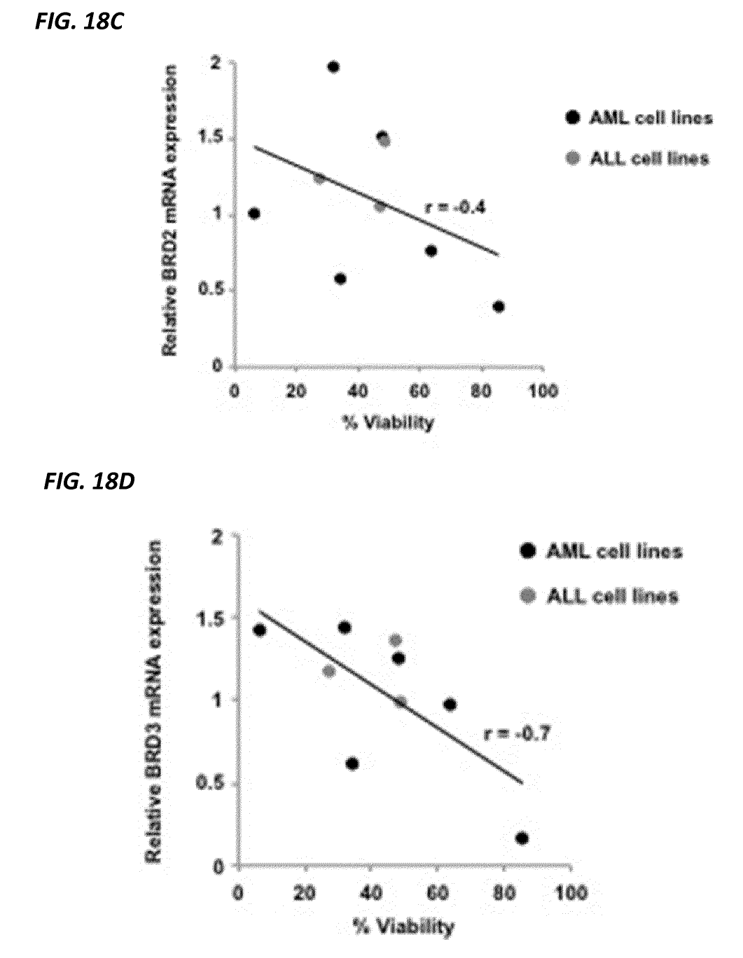

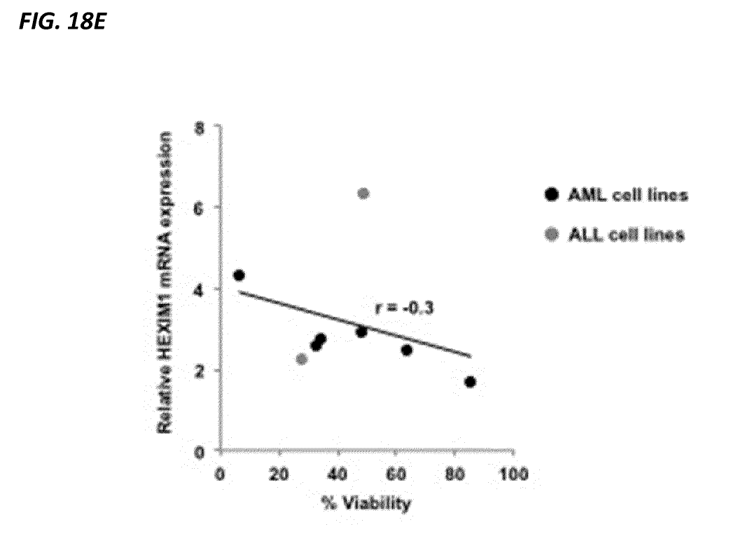

Of note, baseline mRNA expression levels of c-MYC, BRD2, BRD3, BRD4 and HEXIM1 did not significantly correlate with compound (1-1)-induced loss of viability in any of the AML or ALL cell lines analyzed (FIGS. 18A-18E).

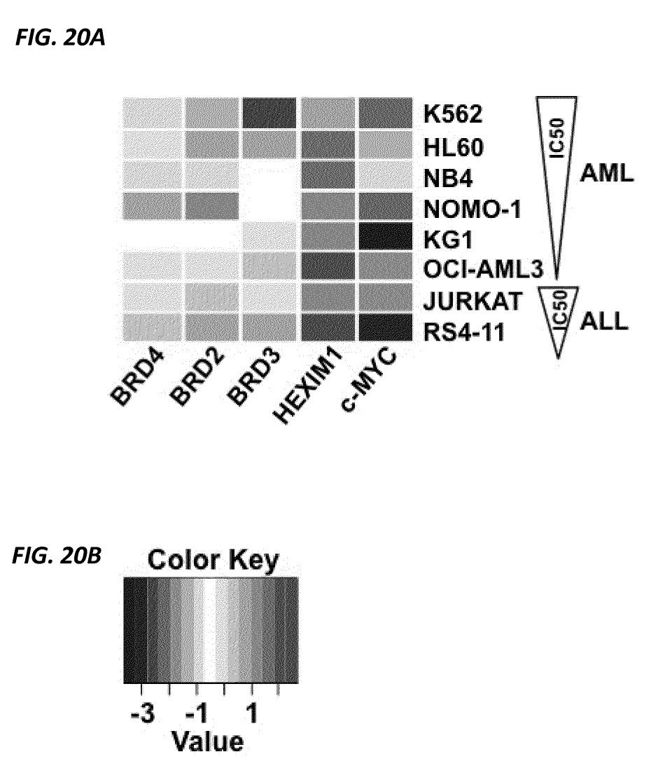

Interestingly, it was not possible to identify any correlations between reduction of cell viability or induction of apoptosis and c-MYC, BRD2/3/4 and HEXIM1 expression levels in cell lines exposed to compound (1-1). The only consistent gene expression modulation induced by compound (1-1) was c-MYC downregulation and HEXIM1 upregulation (FIG. 20) as reported with other BET inhibitors.

### Example 10: Ex Vivo Effects of Compound (1-1) in Leukemic Patient-Derived Samples

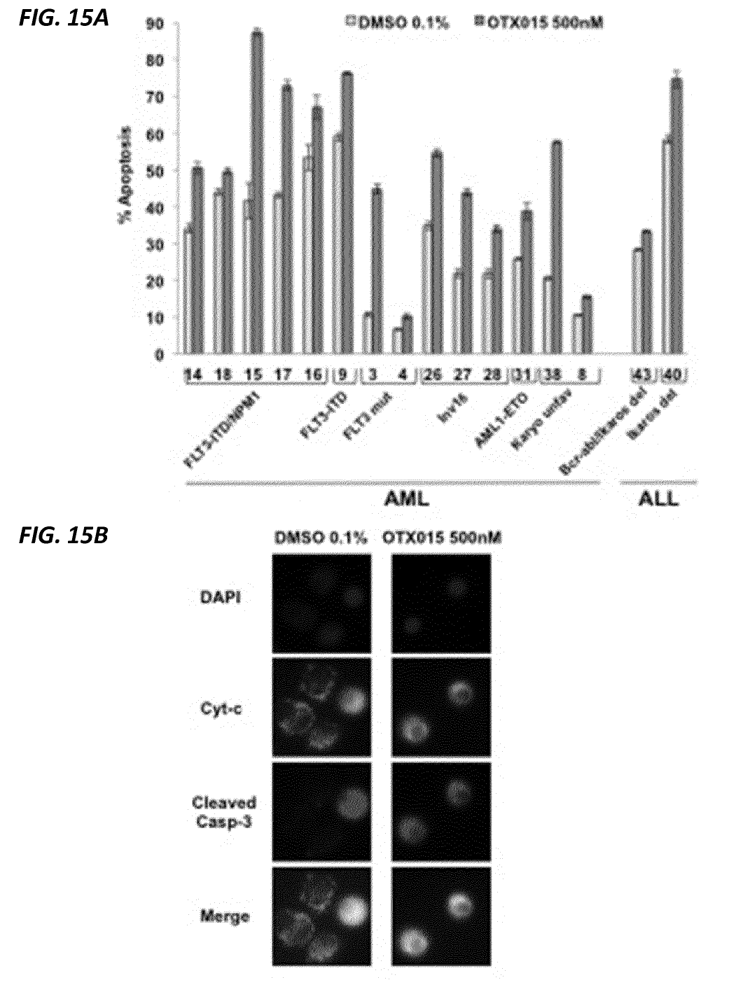

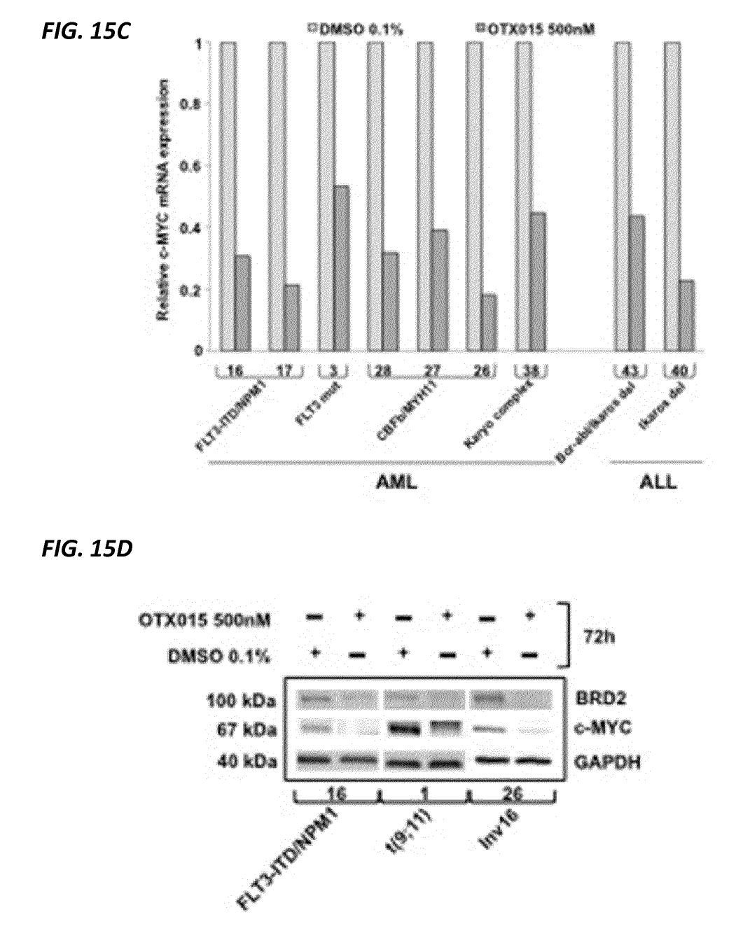

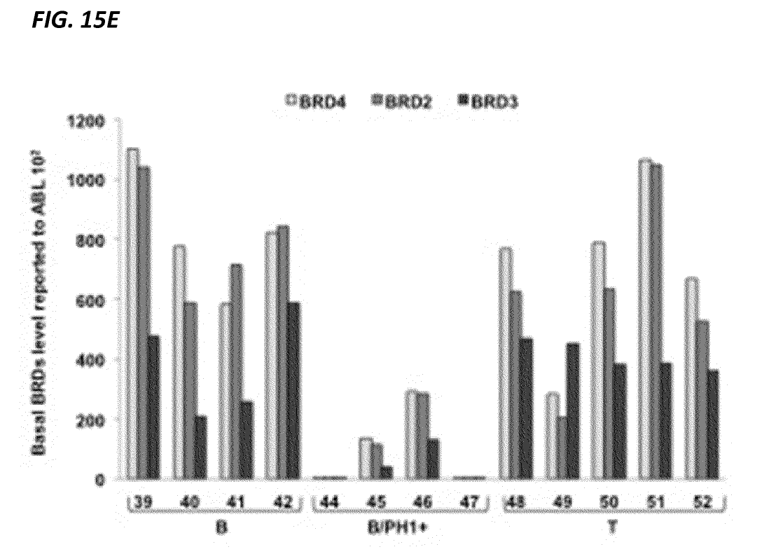

Apoptosis, mRNA and protein expression were evaluated in BM mononuclear cells obtained from representative newly diagnosed or relapsed ALL and AML patients, see Table 7, treated in the context of an ongoing Phase Ib study with compound (1-1) and for whom sufficient material for analysis was available. Apoptosis induction by exposure to 500 nM compound (1-1) for 72 h was variable among the patient samples tested (FIG. 15A). BM cells from 8 of 14 AML patients showed increased apoptosis ranging from 35-90% with compound (1-1) compared to control-treated cells (patients 3, 15, 17, 26, 27, 28, 31 and 38), while no or a mild increase in apoptosis was observed after compound (1-1) exposure in 6 of 14 patients (patients 4, 8, 9, 14, 16 and 18). BM cells from the two ALL patients tested showed no or a mild increase in apoptosis (patients 40 and 43). In line with our observations in cell lines, compound (1-1) also induced activation of caspase-3 and mitochondrial cytochrome c release in samples analyzed from three AML patients (FIG. 15B).

After treatment with 500 nM compound (1-1) for 48 h, c-MYC mRNA expression was downregulated in the seven AML samples and two ALL samples (FIG. 15C) evaluated. In three primary AML samples, c-MYC protein expression had clearly decreased after 72 h with 500 nM compound (1-1), as did BRD2 protein expression (FIG. 15D).

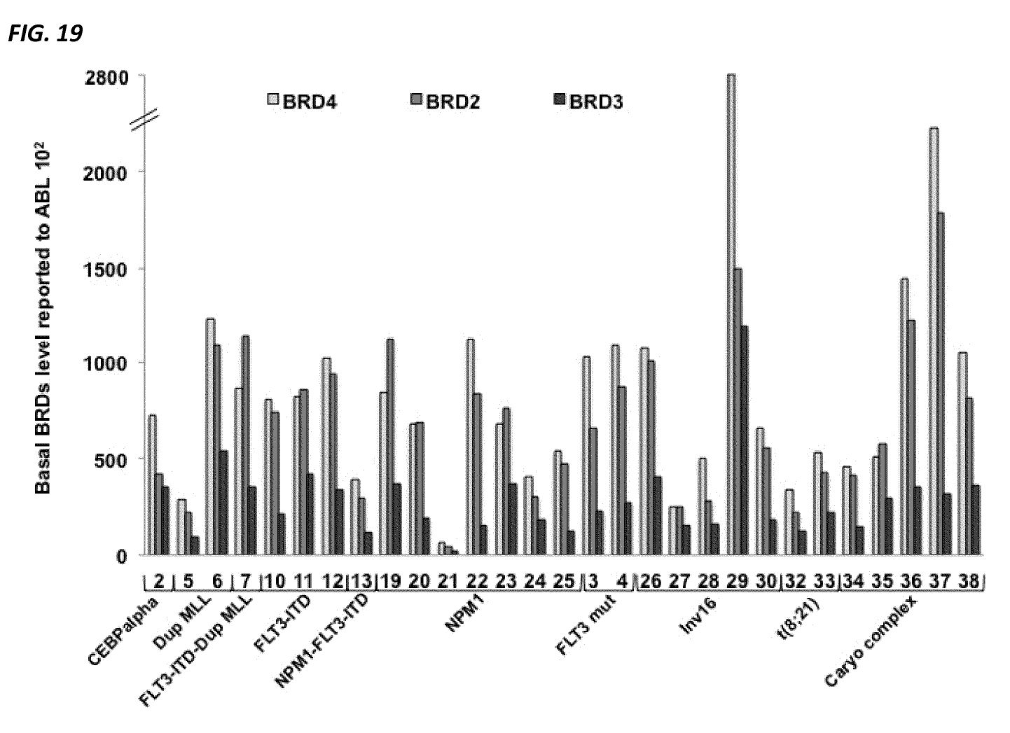

The basal BRD2/3/4 gene expression was studied in 38 AML and 14 ALL patient samples of various subtypes. As observed in cell lines, gene expression levels were highly variable across AML and ALL subtypes with the lowest expression in bcr-abl rearranged ALL samples (FIGS. 15E, 19).

TABLE 7
ALL and AML patient characteristics

| Patient | | | | Molecular | | | |
|---|---|---|---|---|---|---|---|
| No | Gender | Diagnosis | Karyotype | Biology | Apoptosis | mRNA | Protein |
| 1 | M | AML 2 | 46; XY; t(9; 11) | MLLT3-MLL | no | no | yes |
| 2 | M | AML 2 | 46; XY | CEBP alpha | no | yes | no |
| 3 | F | AML 1 | 46; XX | FLT3 mut | yes | no | no |
| 4 | M | sAML | 46; XY | FLT3 mut | yes | no | no |
| 5 | M | AML 2 | 46; XY | dup MLL | no | yes | no |
| 6 | F | AML 1 | 46; XX | dup MLL | no | yes | no |
| 7 | M | AML 5a | 46; XY | FLT3 ITD/ | no | yes | no |
| | | | | dup MLL | | | |
| 8 | M | sAML | 46; XY; | FLT3 ITD/ | yes | no | no |
| | | | del(3)(q?), −7 | EVI1 | | | |
| 9 | M | AML 5a | 46; XY | FLT3 ITD | yes | no | no |
| 10 | F | AML 2 | 46; XX | FLT3 ITD | no | yes | no |
| 11 | M | AML | 46; XY | FLT3 ITD | no | yes | no |
| 12 | F | AML | 47; XX; der(10)(?) | FLT3 ITD | no | yes | no |
| 13 | F | AML 1 | 46; XX | NPM1 + | no | yes | no |
| | | | | FLT3 ITD | | | |
| 14 | F | sAML | 46; XX | NPM1 + | yes | no | no |
| | | | | FLT3 ITD | | | |
| 15 | M | AML 4 | 46; XY | NPM1 + | yes | no | no |
| | | | | FLT3 ITD | | | |
| 16 | M | AML 5 | 46; XY | NPM1 + | yes | no | yes |
| | | | | FLT3 ITD | | | |
| 17 | M | AML 4 | 46; XY | NPM1 + | yes | no | no |
| | | | | FLT3 ITD | | | |
| 18 | M | AML 2 | 46; XY | NPM1 + | yes | no | no |
| | | | | FLT3 ITD | | | |
| 19 | F | AML 2 | 46; XX | NPM1 | no | yes | no |
| 20 | F | AML 2 | 46; XX | NPM1 | no | yes | no |
| 21 | M | AML 4 | 46; XY | NPM1 | no | yes | no |
| 22 | M | AML 4 | 46; XY | NPM1 | no | yes | no |
| 23 | F | AML 4 | 46; XX | NPM1 | no | yes | no |
| 24 | M | AML 1 | 46; XY | NPM1 | no | yes | no |
| 25 | M | AML 2 | 46; XY | NPM1 | no | yes | no |
| 26 | F | AML 4eo | 46; XX; | CBFb/ | yes | yes | yes |
| | | | inv(16)(p13q22) | MYH11 | | | |
| 27 | F | AML 4eo | 46; XX; | CBFb/ | yes | yes | no |
| | | | inv(16)(p13q22) | MYH11 | | | |
| 28 | M | AML 4eo | 46; XY; | CBFb/ | yes | yes | no |
| | | | inv(16)(p13q22) | MYH11 | | | |
| 29 | M | AML 4eo | 46; XY; | CBFb/ | no | yes | no |
| | | | inv(16)(p13q22) | MYH11 | | | |
| 30 | F | AML 4eo | 46; XX; | CBFb/ | no | yes | no |
| | | | inv(16)(p13q22) | MYH11 | | | |
| 31 | F | AML 2 | 46; XX; t(8; 21) | AML1-ETO | yes | no | no |
| 32 | M | AML 2 | 46; XY; t(8; 21) | AML1-ETO | no | yes | no |
| 33 | M | AML 2 | 46; XY; t(8; 21) | AML1-ETO | no | yes | no |
| 34 | F | AML | complex | ND | no | yes | no |
| 35 | F | AML | complex | ND | no | yes | no |
| 36 | M | AML | complex | ND | no | yes | no |
| 37 | M | AML | complex | ND | no | yes | no |
| 38 | F | AML 5 | complex | ND | yes | yes | no |
| 39 | F | ALL-B | complex | Ikaros del | no | yes | no |
| 40 | F | ALL-B | complex | Ikaros del | yes | yes | no |
| 41 | M | ALL-B | 46; XY | ND | no | yes | no |
| 42 | F | ALL-B | 46; XX | ND | no | yes | no |
| 43 | M | ALL-B | 46; XY; t(9; 22) | BCR-ABL | yes | no | yes |
| 44 | M | ALL-B | 46; XY; t(9; 22) | BCR-ABL | no | yes | no |
| 45 | F | ALL-B | 46; XX; t(9; 22) | BCR-ABL | no | yes | no |
| 46 | M | ALL-B | 46; XY; t(9; 22) | BCR-ABL | no | yes | no |
| 47 | M | ALL-B | 46; XY; t(9; 22) | BCR-ABL | no | yes | no |
| 48 | M | ALL-T | 46; XY; | HOX11L2 | no | yes | no |
| | | | del(7)(p?) | | | | |
| 49 | F | ALL-T | complex | ND | no | yes | no |
| 50 | F | ALL-T | complex | ND | no | yes | no |
| 51 | M | ALL-T | complex | ND | no | yes | no |
| 52 | M | ALL-T | ND | Calm/Af10 | no | yes | no |

F, female;
M, male;
ND, not determined

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

1-32. (canceled)

33. A method of treating acute myeloid leukemia or acute lymphoid leukemia in a mammal comprising administering a pharmaceutically acceptable amount of a compound which is (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide dihydrate, or (S)-2-[4-(4-chlorophenyl)-2,3,9-trimethyl-6H-thieno[3,2-f][1,2, 4]triazolo[4,3-a][1,4]diazepin-6-yl]-N-(4-hydroxyphenyl)acetamide, or a pharmaceutically acceptable salt thereof wherein expression of HEXIM1 is upregulated after administration of the compound wherein the compound is formed as a solid dispersion comprising an amorphous compound and a pharmaceutically acceptable polymer which is hydroxypropylemethylcellulose acetate succinate (HPMCAS) having a compound to HPMCAS weight ratio of 1:3 to 1:1.

34. The method according to claim 33, wherein the compound is (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide dihydrate.

35. The method according to claim 33, wherein the compound is (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl) acetamide.

36. The method according to claim 33, wherein the solid dispersion exhibits an X-ray powder diffraction pattern substantially free of diffraction lines associated with crystalline compound.

37. The method according to claim 36, wherein the solid dispersion exhibits a single glass transition temperature (Tg) inflection point ranging from about 130° C. to about 140° C.

38. The method according to claim 36, wherein the solid dispersion exhibits an X-ray powder diffraction pattern substantially free of diffraction lines associated with crystalline compound of (S)-2-[4-(4-chlorophenyl)-2,3,9-trimethyl-6H-thieno[3,2-f][1,2,-4]triazolo[4,3-a][1,4]diazepin-6-yl]-N-(4-hydroxyphenyl)acetamide dihydrate.

39. The method according to claim 33, wherein c-MYC RNA levels are downregulated.

40. The method according to claim 33, wherein BRD2 mRNA levels are downregulated.

41. The method according to claim 33, wherein BRD4 mRNA levels are downregulated.

42. The method according to claim 33, wherein the acute lymphoid leukemia is a MLL-rearranged acute lymphoid leukemia.

43. The method according to claim 42, wherein the MLL-rearranged acute lymphoid leukemia is characterized by the fusion of the MLL gene to the AF4 (MLLT3) gene.

44. The method according to claim 32, wherein the acute myelogenous leukemia is a MLL-rearranged acute myelogenous leukemia.

45. The method according to claim 44, wherein the MLL-rearranged acute myelogenous leukemia is characterized by the fusion of the MLL gene to the AF9 (MLLT3) gene.

46. The method according to claim 32, wherein the acute myelogenous leukemia is a NPM1-mutated acute myelogenous leukemia.

47. The method according to claim 32, wherein the acute myelogenous leukemia is BCR-ABL associated acute myeloic leukemia.

48. The method according to claim 32, wherein the acute lymphatic leukemia is BCR-ABL associated acute lymphatic leukemia.

49. The method according to claim 32, wherein the acute myelogenous leukemia harbors a PML-RARα fusion gene.

50. The method according to claim 32, wherein the acute myelogenous leukemia is a NRAS mutated acute myelogenous leukemia.

51. The method according to claim 32, wherein the acute myelogenous leukemia harbors a OP2-FGFR1 fusion gene.
